(19) United States
(12) Patent Application Publication
(10) Pub. No.: US 2023/0210943 A1
(43) Pub. Date: Jul. 6, 2023

(54) TREATMENT OF NON-ALCOHOLIC FATTY LIVER DISEASE

(71) Applicant: PEMI31 THERAPEUTICS INC., Calgary (CA)

(72) Inventor: Edward Redmond O'BRIEN, Calgary (CA)

(21) Appl. No.: 17/927,728

(22) PCT Filed: May 28, 2021
(86) PCT No.: PCT/CA2021/050728

§ 371 Date: Nov. 24, 2022

Related U.S. Application Data
(60) Provisional application No. 63/031,640, filed on May 29, 2020.

(51) Int. Cl. A61K 38/17; C07K 16/18; A61P 1/16

(52) U.S. Cl. CPC A61K 38/1709; C07K 16/18; A61P 1/16

(57) ABSTRACT

Disclosed are methods for the treatment of non-fatty liver disease (NAFLD). The methods involve the administration to a subject in need thereof of a pharmaceutical formulation comprising an HSP27 polypeptide or immunologically equivalent portion thereof, or an anti-HSP27 antibody or a functional anti-HSP27 antibody fragment, or a mixture of an HSP27 polypeptide or immunologically equivalent portion thereof, and an anti-HSP27 antibody or a functional anti-HSP27 antibody fragment.

### RELATED APPLICATION

This application claims the benefit of U.S. Provisional Patent Application No. 63/031,640 filed May 29, 2020; the entire contents of Patent Application No. 63/031,640 are hereby incorporated by reference.

### FIELD OF THE DISCLOSURE

The methods and systems disclosed herein generally relate to liver diseases and methods of treatment thereof. More specifically, the present disclosure relates to methods for treating non-alcoholic fatty liver disease.

### BACKGROUND OF THE DISCLOSURE

The following paragraphs are provided by way of background to the present disclosure.

Non-alcoholic fatty liver disease (NAFLD) is a commonly occurring liver disorder which develops in the absence of secondary causes, notably excessive alcohol consumption. Standard clinical observations associated with NAFLD include fatty accumulation in liver cells, a pathological condition also referred to as simple hepatic steatosis, or non-alcoholic fatty liver (NAFL), characterized by the liver having a fat content of more than 5%. Furthermore, it is known that patients with NAFLD can proceed to develop a more serious inflammatory form of NAFLD, known as non-alcoholic steatohepatitis (NASH). In addition to hepatic steatosis, patients with NASH progress to exhibit liver fibrosis (early stage scarring) and can further develop liver cirrhosis (late stage scarring), and cancer, notably hepatocellular carcinoma (HCC), which develops in about 13% of patients with NASH exhibiting cirrhosis and fibrosis.

It is estimated that there are 67 million adults in the United States with simple hepatic steatosis, and 13 million adults in the United States with NASH exhibiting fibrosis and/or cirrhosis. Globally the NASH patient population may exceed 500 million patients. Furthermore, lifetime costs for patients with NASH in the United States are estimated to be $222.6 billion US Dollars (Younossi, Z. M. et al., 2019, Hepatology, 69(2): 564-572). Moreover, patients with NAFLD and NASH experience a significantly reduced health-related quality of life (Sayiner, M. et al., 2016, Hepatology, BMJ Open Gastro 2016; 3:e000106).

As most patients with NAFLD remain asymptomatic, NAFLD is most commonly diagnosed by laboratory tests showing high liver aminotransferases or hepatic imaging showing excess fat accumulation. Liver biopsy is currently the standard method for diagnosis of NASH. Furthermore, biopsy confirmed endpoints are required by the Food and Drug Administration (FDA) in the United States, and by the European Medicines Agency (EMA) in order to obtain approval for NASH drug candidates.

Patients with NAFLD or NASH may be prescribed increased physical activity, dietary improvements, or medications to modulate blood glucose, blood pressure, and blood lipids, with the goal of reducing weight (Chalasani et al., 2018, Hepatology, doi: 10.1002/hep.29367). However, the degree of weight loss required for histologic improvement of NASH may be difficult to achieve and even harder to sustain for patients. In some cases, bariatric surgery remains the only effective weight-loss therapy and has also demonstrated gains in cardiovascular outcomes, the leading cause of premature mortality in patients with NAFLD. For patients with liver biopsy-proven NASH and fibrosis score but without diabetes, vitamin E (800 international units per day) may be used, although evidence of efficacy is limited. In patients with NASH and diabetes, metformin and/or pioglitazone/liraglutide may be prescribed. When severe enough, liver cirrhosis can compromise normal liver function, and the need for a liver transplant may arise. However, despite the sizable patient population and the severity of NAFLD and NASH, no specific FDA approved pharmaceutical treatments for NAFLD or NASH are currently available. Thus, there is an interest in the development of pharmaceutical products to treat NAFLD and NASH.

Accordingly, there exists, a need in the art for improved methods for the treatment of non-alcoholic fatty liver disease. In particular, there is a need in the art for pharmaceutical therapeutic treatments for NAFLD and NASH.

### SUMMARY OF THE DISCLOSURE

The following paragraphs are intended to introduce the reader to the more detailed description, not to define or limit the claimed subject matter of the present disclosure.

In one aspect, the present disclosure relates to liver disease.

In another aspect, the present disclosure relates to methods to treat non-alcoholic fatty liver disease.

Accordingly, in one aspect, the present disclosure provides, in at least one embodiment, a method for treating non-alcoholic fatty liver disease, the method comprising administering to a human subject in need thereof a pharmaceutical formulation comprising at least one of (i) an HSP27 polypeptide or an immunologically equivalent portion thereof, or (ii) an anti-HSP27 antibody or functional antibody fragment thereof, wherein the pharmaceutical formulation is administered in an effective amount to treat non-alcoholic fatty liver disease in the subject.

In at least one embodiment, the HSP27 polypeptide or the immunologically equivalent portion thereof can be a polypeptide encoded by a nucleic acid sequence selected from the nucleic acid sequences consisting of:
(a) SEQ.ID NO: 1; SEQ.ID NO: 15; SEQ.ID NO: 16; SEQ.ID NO: 17; SEQ.ID NO: 18; SEQ.ID NO: 19; SEQ.ID NO: 20; SEQ.ID NO: 21; SEQ.ID NO: 22; and SEQ.ID NO: 23;
(b) a nucleic acid sequence that is substantially identical to SEQ.ID NO: 1; SEQ.ID NO: 15; SEQ.ID NO: 16; SEQ.ID NO: 17; SEQ.ID NO: 18; SEQ.ID NO: 19; SEQ.ID NO: 20; SEQ.ID NO: 21; SEQ.ID NO: 22; and SEQ.ID NO: 23;
(c) a nucleic acid sequence that is substantially identical to SEQ.ID NO: 1; SEQ.ID NO: 15; SEQ.ID NO: 16; SEQ.ID NO: 17; SEQ.ID NO: 18; SEQ.ID NO: 19; SEQ.ID NO: 20; SEQ.ID NO: 21; SEQ.ID NO: 22; and SEQ.ID NO: 23 but for the degeneration of the genetic code;
(d) a nucleic acid sequence that is complementary to SEQ.ID NO: 1; SEQ.ID NO: 15; SEQ.ID NO: 16; SEQ.ID NO: 17; SEQ.ID NO: 18; SEQ.ID NO: 19; SEQ.ID NO: 20; SEQ.ID NO: 21; SEQ.ID NO: 22; and SEQ.ID NO: 23;
(e) a nucleic acid sequence encoding a polypeptide having the amino acid sequence set forth in SEQ.ID NO: 2, SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14;
(f) a nucleic acid sequence that encodes an immunologically equivalent functional variant of the amino acid sequence set forth in SEQ.ID NO: 2, SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14; and
(g) a nucleic acid sequence that hybridizes under stringent conditions to any one of the nucleic acid sequences set forth in (a), (b), (c), (d), (e), or (f).

In at least one embodiment, the immunologically equivalent portion of the HSP27 polypeptide can comprise at least 30, at least 40, at least 50, at least 60, at least 70, at least 80, at least 90, at least 100, or 105 consecutive amino acids of the 105 N-terminal amino acid residue portion of SEQ.ID NO: 2.

In at least one embodiment, the immunologically equivalent portion of the HSP27 polypeptide can be a polypeptide comprising at least two, at least three, at least four, at least five, at least six, at least seven at least eight, or all nine of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14.

In at least one embodiment, the immunologically equivalent portion of the HSP27 polypeptide can be a polypeptide comprising (a) (i) at least two of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, and (ii) at least 30 consecutive amino acids of the 105 N-terminal amino acid residue portion of SEQ.ID NO: 2, or (b) (iii) at least three of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and (iv) SEQ.ID NO: 14, and at least 50 consecutive amino acids of the 105 N-terminal amino acid residue portion of SEQ.ID NO: 2.

In at least one embodiment, the immunologically equivalent portion of the HSP27 polypeptide can comprise a peptide selected from SEQ.ID NO; 25; SEQ.ID NO: 26; SEQ.ID NO; 27; SEQ.ID NO: 28; SEQ.ID NO; 29; SEQ.ID NO: 30; SEQ.ID NO; 31; SEQ.ID NO: 32; and SEQ.ID NO: 33.

In at least one embodiment, the anti-HSP27 antibody can be a polyclonal anti-HSP27 antibody.

In at least one embodiment, the anti-HSP27 antibody can be a monoclonal anti-HSP27 antibody.

In at least one embodiment, the anti-HSP27 antibody can be an antibody belonging to the IgG class of antibodies.

In at least one embodiment, the anti-HSP27 antibody can be an antibody binding to at least one HSP27 epitope selected from: SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14.

In at least one embodiment, the pharmaceutical formulation can comprise an HSP27 polypeptide or an immunologically equivalent portion thereof and an adjuvant.

In at least one embodiment, the pharmaceutical formulation can comprise an HSP27 polypeptide or an immunologically equivalent portion thereof and an adjuvant, wherein the pharmaceutical formulation does not include the anti-HSP27 antibody or a functional antibody fragment thereof.

In at least one embodiment, the pharmaceutical formulation can comprise an HSP27 polypeptide or an immunologically equivalent portion thereof and an adjuvant, and not the anti-HSP27 antibody or a functional antibody fragment thereof, the HSP27 polypeptide or immunologically equivalent portion thereof eliciting an immune response in the subject and the production of native anti-HSP27 antibodies in the blood serum of the subject.

In at least one embodiment, the pharmaceutical formulation can comprise an HSP27 polypeptide or an immunologically equivalent portion thereof and an adjuvant, and not the anti-HSP27 antibody or a functional antibody fragment thereof, the HSP27 polypeptide or immunologically equivalent portion thereof eliciting an immune response in the subject and the production of native anti-HSP27 antibodies in the blood serum of the subject, the HSP27 polypeptide or immunologically equivalent portion thereof and native anti-HSP27 antibodies forming polypeptide/antibody complexes, wherein said complexes bind to the cell membrane of macrophage cells or liver cells in the subject to thereby trigger an anti-inflammatory response.

In at least one embodiment, the pharmaceutical formulation can comprise an anti-HSP27 antibody, or a functional fragment thereof, wherein the formulation does not include the HSP27 polypeptide or an or immunologically equivalent portion thereof.

In at least one embodiment, the pharmaceutical formulation can comprise an anti-HSP27 antibody, or a functional fragment thereof, and does not include the HSP27 polypeptide or immunologically equivalent portion thereof, wherein upon administration a polypeptide/antibody complex is formed between the anti-HSP27 antibody or a functional fragment thereof and the native HSP27 polypeptide of the subject in the blood serum of the subject, and wherein said complex binds to the cell membrane of macrophage cells or liver cells in the subject to thereby trigger an anti-inflammatory response.

In at least one embodiment, the pharmaceutical formulation can comprise an HSP27 polypeptide or immunologically equivalent portion thereof and an anti-HSP27 antibody or anti-HSP27 functional antibody fragment thereof, the HSP27 polypeptide or immunologically equivalent portion thereof and the native anti-HSP27 antibodies forming polypeptide/antibody complexes or polypeptide/anti-HSP27 functional antibody fragment complexes, the complexes being present in the formulation, or being formed in vivo in the blood serum of the subject upon administration, wherein said complexes bind to the cell membrane of macrophage cells or liver cells in the subject to thereby trigger an anti-inflammatory response.

In at least one embodiment, the pharmaceutical formulation can comprise an anti-HSP27 antibody, or a functional fragment thereof, and does not include the HSP27 polypeptide or immunologically equivalent portion thereof, wherein upon administration a complex is formed between the anti-HSP27 antibody or a functional fragment thereof, and the native HSP27 of the subject in the blood serum of the subject, wherein the formed complex limits the interaction of circulating oxidized low density lipoprotein (oxLDL) with at least one of liver cell scavenger receptors SR-A1 or CD-36, to thereby restrict uptake of the circulating oxLDL by the liver cells of the subject.

In at least one embodiment, the pharmaceutical formulation can comprise an HSP27 polypeptide or immunologically equivalent portion thereof and an anti-HSP27 antibody or anti-HSP27 functional antibody fragment thereof, the HSP27 and native anti-HSP27 antibodies forming polypeptide/antibody complexes or polypeptide/anti-HSP27 functional antibody fragment complexes, the complexes being present in the formulation, or being formed in vivo in the in the blood serum of the subject upon administration, wherein the interaction of circulating oxLDL with at least one of liver cell scavenger receptors SR-A1 or CD-36 is reduced, to thereby restrict uptake of the circulating oxLDL by the liver cells of the subject.

In at least one embodiment, the pharmaceutical formulation can comprise the HSP27 polypeptide or immunologically equivalent portion thereof in a concentration ranging from about 100 μg/ml to about 10 mg/ml, or the anti-HSP27 antibody or functional antibody fragment thereof in a concentration ranging from about 25 mg/ml to about 200 mg/ml.

In at least one embodiment, the pharmaceutical formulation can comprise a HSP27 polypeptide or an immunologically equivalent portion thereof, and a dose ranging from about 100 μg to about 1 mg thereof can be administered to the human subject, or the pharmaceutical formulation can comprise an anti-HSP27 antibody or a functional antibody fragment thereof, and a dose ranging from about 100 mg to about 5 g can be administered to the human subject.

In at least one embodiment, the pharmaceutical formulation further can comprise a pharmaceutically acceptable carrier, excipient or diluent.

In at least one embodiment, the pharmaceutical formulation can be administered parenterally.

In at least one embodiment, the non-alcoholic fatty liver disease can be non-alcoholic steatohepatitis.

In at least one embodiment, the non-alcoholic steatohepatitis can be characterized by hepatic fibrosis or hepatic cirrhosis.

In another aspect, the present disclosure further provides, in at least one embodiment, a use of at least one of (i) a HSP27 polypeptide or an immunologically equivalent portion thereof, or (ii) an anti-HSP27 antibody or functional antibody fragment thereof, in the manufacture of a medicament for the treatment of non-alcoholic fatty liver disease in a human subject in need thereof.

In another aspect, the present disclosure further provides, in at least one embodiment, a use of a pharmaceutical formulation comprising at least one of (i) a HSP27 polypeptide or immunologically equivalent portion thereof, or (ii) an anti-HSP27 antibody or functional antibody fragment thereof to treat non-alcoholic fatty liver disease in a human subject in need thereof.

In another aspect, the present disclosure provides, in at least one embodiment, a pharmaceutical formulation comprising at least one of (i) a HSP27 polypeptide or immunologically equivalent portion thereof, or (ii) an anti-HSP27 antibody or functional antibody fragment thereof, for use in treating non-alcoholic fatty liver disease in a human subject in need thereof.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIGS. 1A, 1B, 1C, 1D, 1E, and 1F depict graphs representing results from selected experiments described herein, notably (i) in FIG. 1A: a bar graph representing results of a cell surface binding assay using THP-1 macrophage cells; (ii) in FIG. 1B: a bar graph representing results of an in vitro binding assay using THP-1 macrophage cell lysates; (iii) in FIG. 1C: a bar graph representing results of NF-κB SEAP reporter gene assay of THP1-XBlue™ macrophage monocytes treated for 24 hr with different combinations of rHSP27 (1 μg/ml), rC1 (1 μg/ml), and PAb (5 μg/ml); (iv) in FIG. 1D: a bar graph representing results of a NF-κB reporter gene assay using THP1 XBlue™ macrophage with different combinations of rHSP27 (1 μg/ml) and PAb (5 μg/ml) and LPS (10 and 100 ng/ml); (v) in FIG. 1E: a graph representing results of a competitive binding experiment between LPS and rHSP27 or [rHSP27+PAb] complex; and (vi) in FIG. 1F: a bar graph representing results of a competitive binding experiment detecting cytokine (IL-1β and IL-10) production by THP-1 macrophage cells treated with LPS with different combinations of rHSP27, rC1, and Pab.

FIGS. 2A, 2B, 2C, 2D, 2E, and 2F depict graphs representing results from selected experiments described herein, notably (i) in FIG. 2A: a bar graph representing results of an in vitro binding assay using THP-1 macrophage cell lysates; (ii) in FIG. 2B: a bar graph representing results of another in vitro binding assay using THP-1 macrophage cell lysates; (iii) in FIG. 2C: a graph representing results of a binding competition assay detecting OxLDL interaction with SR-AI receptor as a function of HSP27 concentration and comparing HSP27 alone and [HSP27+PAb] complex; (iv) in FIG. 2D: a graph representing results of a binding competition assay detecting OxLDL interaction with CD36 receptor as a function of HSP27 concentration and comparing HSP27 alone and [HSP27+PAb] complex; (v) in FIG. 2E: a graph representing results of a binding competition assay detecting HSP27 binding to SR-AI receptor as a function of OxLDL concentration and comparing HSP27 alone and [HSP27+Pa] complex; and (vi) in FIG. 2F: a graph representing results of a binding competition assay detecting HSP27 binding to CD36 receptor as a function of OxLDL concentration and comparing HSP27 alone and [HSP27+Pa] complex.

FIGS. 3A, 3B, and 3C depict graphs representing results from selected experiments described herein, notably: (i) in FIG. 3A: flow cytometric graphs of Dil-oxLDL uptake in HEK Blue™ cells expressing SR-AI (left panels), CD36 (middle panels) or Null (right panels). Cells were treated with combinations of rHSP27, rC1 and PAb (as indicated in each individual panel), and the following parameters were recorded: a) percentage of cells positive for Dil-oxLDL fluorescence (as indicated in each panel); and b) the mean fluorescence intensity for Dil-oxLDL uptake per treatment group (as indicated in the top right corner of each panel); (ii) in FIG. 3B: flow cytometric graphs representing results of a fluorescence activating sorting assay (FACS) study similar to (i) to assay Dil-oxLDL uptake but in THP-1 macrophages; and in FIG. 3C: a bar graph obtained using a plate reader assay to evaluate Dil-oxLDL uptake in THP-1 macrophages.

FIGS. 4A, 4B, 4C, 4D and 4E depict photographs documenting certain experimental results described herein obtained using certain laboratory diagnostic devices, (4A, 4B, 4D, and 4E) and a diagram comprising certain information relating to a polypeptide (FIG. 4C), notably, (i) in FIG. 4A: 8 spot blot membranes, each membrane containing a series of HSP27 peptides, the peptides together spanning the full length HSP27 and reacted with human polyclonal anti-HSP27 antibodies of four healthy individuals (“CON”) and four cardiovascular disease patients (“CVD”), wherein reacting HSP27 peptides light up, and as such represent HSP27 epitopes; (ii) in FIG. 4B: 4 spot blot membranes each membrane containing a series of HSP27 peptides, the peptides together spanning the full length HSP27 and reacted with human anti-HSP27 polyclonal antibodies of one healthy individual (“CON”) and one cardiovascular disease patient (“CVD”), wherein the reaction was conducted in the presence (“+”) or absence (“−”) of excess (exogenous) HSP27; (iii) in FIG. 4C: a diagram showing a HSP27 polypeptide and certain peptide sequences corresponding with HSP27 epitopes identified based on the results shown in FIGS. 4A, 4B and 4D; (iv) in FIG. 4D: a spot blot membrane containing a series of HSP27 peptides, the peptides together spanning the full length HSP27 and reacted with rabbit anti-HSP27 polyclonal antibodies, thereby validating the fidelity of this antibody for in vitro experiments; and (v) in FIG. 4E: four western blots, each comprising various quantities of HSP 27 (10 ng, 100 ng and 1 μg) and HSP 25 (100 ng and 1 μg), reacted with goat polyclonal anti-HSP27 antibodies in the presence (“+rHSP27”) and absence (“alone”) of HSP27, and reacted with rabbit polyclonal anti-HSP27 antibodies in the presence (“+rHSP27”) and absence (“alone”) of HSP27. It is noted in that in FIG. 4C noted amino acid sequence #4 corresponds with SEQ.ID. NO: 15, noted amino acid sequence #7 corresponds with SEQ.ID. NO: 16, noted amino acid sequence #12 corresponds with SEQ.ID. NO: 17, noted amino acid sequence #13 corresponds with SEQ.ID. NO: 18, noted amino acid sequence #16 corresponds with SEQ.ID. NO: 19, noted amino acid sequence #17 corresponds with SEQ.ID. NO: 20, noted amino acid sequence #18 corresponds with SEQ.ID. NO: 21, noted amino acid sequence #22 corresponds with SEQ.ID. NO: 22, and noted amino acid sequence #45 corresponds with SEQ.ID. NO: 23.

FIGS. 5A, 5B, 5C, 5D, 5E, 5F, 5G and 5H depict microscopic images (FIGS. 5A, 5C and 5E) and graphs (FIGS. 5B, 5D, 5F, 5G, and 5H) representing results from selected experiments described herein, notably microscopic images of liver tissues obtained from mice immunized with rC1 (HSP27) and the mouse ortholog thereof (HSP25), treated and visualized using various stains and reagents, notably hematoxylin & eosin stain (FIG. 5A), macrophage (Mac-2) stain (brown) (FIG. 5C), picrosirius red stain light microscopy (FIG. 5E, left images), and polarized light microscsopy (FIG. 5E, right images), and graphs showing a quantitative comparison between mice immunized with rC1 (HSP27) and the mouse ortholog thereof (HSP25) of hepatic lesions (FIG. 5B), the presence of macrophage cells (FIG. 5C), the presence of hepatic collagen (FIG. 5F), expression of interleukin 1-beta (IL-1β) and expression of tumor necrosis factor alpha (TNFα) (see: FIG. 5G).

The figures together with the following detailed description make apparent to those skilled in the art how the disclosure may be implemented in practice.

### DETAILED DESCRIPTION

Various methods, processes or compositions will be described below to provide an example of an embodiment of each claimed subject matter. No embodiment described below limits any claimed subject matter and any claimed subject matter may cover methods, processes or compositions that differ from those described below. The claimed subject matter is not limited to methods, processes or compositions having all of the features of any one method, process or composition described below or to features common to multiple or all of the methods, processes or compositions described below. It is possible that a method, process or composition described below is not an embodiment of any claimed subject matter. Any subject matter disclosed in a method, process or composition described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers.

The term “or” is inclusive unless modified, for example, by “either”. The term “and/or” as used herein means that the listed items are present, or used, individually or in combination. That is, “X and/or Y” is intended to mean X or Y or both, for example. As a further example, “X, Y, and/or Z” is intended to mean X or Y or Z or any combination thereof. In effect, this term means that “at least one of” or “one or more” of the listed items is used or present.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore any range of values described herein is intended to specifically include the limiting values of the range, and any intermediate value or sub-range within the given range, and all such intermediate values and sub-ranges are individually and specifically disclosed (e.g. a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present invention, which is defined solely by the claims.

All publications, patents and patent applications are herein incorporated by reference in their entirety to the same extent as if each individual publication, patent or patent application was specifically and individually indicated to be incorporated by reference in its entirety.

### TERMS AND DEFINITIONS

The terms “nucleic acid”, or “nucleic acid sequence”, as used herein, refer to a sequence of nucleoside or nucleotide monomers, consisting of naturally occurring bases, sugars and intersugar (backbone) linkages. The term also includes modified or substituted sequences comprising non-naturally occurring monomers or portions thereof. The nucleic acids of the present disclosure may be deoxyribonucleic nucleic acids (DNA) or ribonucleic acids (RNA) and may include naturally occurring bases including adenine, guanine, cytosine, thymidine and uracil. The nucleic acids may also contain modified bases. Examples of such modified bases include aza and deaza adenine, guanine, cytosine, thymidine and uracil, and xanthine and hypoxanthine. A sequence of nucleotide or nucleoside monomers may be referred to as a polynucleotide sequence, nucleic acid sequence, a nucleotide sequence or a nucleoside sequence.

The term “polypeptide”, as used herein in conjunction with a reference SEQ.ID NO, refers to any and all polypeptides comprising a sequence of amino acid residues which is (i) substantially identical to the amino acid sequence constituting the polypeptide having such reference SEQ.ID NO, or (ii) encoded by a nucleic acid sequence capable of hybridizing under at least moderately stringent conditions to any nucleic acid sequence encoding the polypeptide having such reference SEQ.ID NO, but for the use of synonymous codons. A sequence of amino acid residues may be referred to as an amino acid sequence, or polypeptide sequence.

The term “nucleic acid sequence encoding a polypeptide”, as used herein in conjunction with a reference SEQ.ID NO, refers to any and all nucleic acid sequences encoding a polypeptide having such reference SEQ.ID NO. Nucleic acid sequences encoding a polypeptide, in conjunction with a reference SEQ.ID NO, further include any and all nucleic acid sequences which (i) encode polypeptides that are substantially identical to the polypeptide having such reference SEQ.ID NO; or (ii) hybridize to any nucleic acid sequences encoding polypeptides having such reference SEQ.ID NO under at least moderately stringent hybridization conditions or which would hybridize thereto under at least moderately stringent conditions but for the use of synonymous codons.

The terms “nucleic acid sequence encoding HSP27”, and “nucleic acid sequence encoding a HSP27 polypeptide”, as may be used interchangeably herein, refer to any and all nucleic acid sequences encoding an HSP27 polypeptide, including, for example, SEQ.ID NO: 1. Nucleic acid sequences encoding a HSP27 polypeptide further include any and all nucleic acid sequences which (i) encode polypeptides that are substantially identical to the HSP27 polypeptide sequences set forth herein; or (ii) hybridize to any HSP27 nucleic acid sequences set forth herein under at least moderately stringent hybridization conditions or which would hybridize thereto under at least moderately stringent conditions but for the use of synonymous codons.

The terms “HSP27 polypeptide”, and “HSP27 protein” as may be used herein, interchangeably, refer to any and all protein comprising a sequence of amino acid residues which is (i) substantially identical to the amino acid sequences constituting any HSP27 polypeptide set forth herein, including, for example, SEQ.ID NO: 2, or (ii) encoded by a nucleic acid sequence capable of hybridizing under at least moderately stringent conditions to any nucleic acid sequence encoding any HSP27 polypeptide set forth herein, but for the use of synonymous codons, or (iii) immunologically equivalent to any HSP27 polypeptide set forth herein.

By the term “substantially identical” it is meant that two amino acid sequences preferably are at least 70% identical, and more preferably are at least 85% or 90% identical, and most preferably at least 95% identical, for example 96%, 97%, 98% or 99% identical. In order to determine the percentage of identity between two amino acid sequences the amino acid sequences of such two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Mol. Biol., 1990:215:403). A particularly preferred method for determining the percentage identity between two polypeptides involves the Clustal W algorithm (Thompson, J. D., Higgines, D. G. and Gibson T. J., 1994, Nucleic Acid Res 22(22): 4673-4680 together with the BLOSUM 62 scoring matrix (Henikoff S. & Henikoff, J. G., 1992, Proc. Natl. Acad. Sci. USA 89: 10915-10919 using a gap opening penalty of 10 and a gap extension penalty of 0.1, so that the highest order match obtained between two sequences wherein at least 50% of the total length of one of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5×sodium chloride/sodium citrate (SSC)/5×Denhardt's solution/1.0% SDS at Tm (based on the above equation) −5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

The term “functional variant”, as used herein in reference to polynucleotides or polypeptides, refers to polynucleotides or polypeptides capable of performing the same function as a noted reference polynucleotide or polypeptide. Thus, for example, a functional variant of the polypeptide set forth in SEQ.ID NO: 2, refers to a polypeptide capable of performing the same function as the polypeptide set forth in SEQ.ID NO: 2. Functional variants include modified a polypeptide wherein, relative to a noted reference polypeptide, the modification includes a substitution, deletion or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine and valine, (iv) asparagine and glutamine, and (v) tryptophan, tyrosine and phenylalanine.

The term “anti-HSP27 antibody”, as used herein refers to an intact antibody that is capable of binding an HSP27 polypeptide with sufficient affinity such that the antibody is useful as a therapeutic or diagnostic agent in targeting the HSP27 polypeptide. Generally, the extent of binding of an anti-HSP27 antibody to an unrelated non-HSP27 polypeptide is less than 10%, as measured, e.g. by a radioimmune assay (RIA). In certain embodiments, an anti-HSP27 antibody has a dissociation constant (Kd) from <1 μM to <0.001 nM, and values therebetween.

The term “immunologically equivalent”, as used herein, refers to a molecule that is capable of eliciting a humoral immune response in the form of the production of native polyclonal antibodies in a subject when administered thereto, wherein the binding specificity to the native polyclonal antibodies is comparable to the specificity of native polyclonal antibodies produced when a reference molecule is administered to the subject. Immunologically equivalent fragments of a reference full length HSP27 polypeptide include HSP27 portions which when administered to a subject elicit a humoral immune response in the form of the production of native antibodies with a specificity to an HSP27 polypeptide which is comparable to the binding specificity for an HSP27 polypeptide of native antibodies obtained when the reference full length HSP27 is administered to the subject. To compare binding specificity between a reference molecule and an immunologically equivalent molecule a radioimmune assay (RIA) may be used, and the extent of binding may be measured. The dissociation constant of an immunologically equivalent molecule is preferably at least 50%, at least 60%, at least 70%, at least 80%, at least 90%, or at least 95% of the dissociation constant of a reference molecule.

The term “functional antibody fragment”, as used herein, refers to a molecule, other than an intact antibody, that comprises a portion of an intact antibody that binds to the antigen to which the intact antibody binds. Thus, for example, an anti-HSP27 functional antibody fragment is an antibody fragment that binds to an HSP27 polypeptide to which an intact anti-HSP27 antibody binds. Examples of functional antibody fragments include, but are not limited to, Fv, Fab, Fab′, Fab′-SH, F(ab)₂, diabodies, single chain antibody molecules (e.g. scFv), and multi-specific antibodies (e.g. bi-specific antibodies) formed from antibody fragments.

The term “intact antibody”, as used herein, refers to an antibody having a structure substantially similar to a native antibody structure, and includes native antibodies, as well as polyclonal antibodies, monoclonal antibodies, human antibodies, humanized antibodies, and chimeric antibodies.

The term “native antibody”, as used herein, refers to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VF1), also known as a variable heavy domain or a heavy chain variable domain, followed by three constant domains (CF11, CF12, and CF13). Similarly, from N- to C-terminus, each light chain has a variable region (VL), also called a variable light domain or a light chain variable domain, followed by a constant light (CL) domain. The light chain of an antibody may be assigned to one of two types, called kappa 00 and lambda (λ), based on the amino acid sequence of its constant domain. The variable region of an antibody heavy or light chain is involved in binding the antibody to antigen. The variable regions of the heavy chain and light chain (VF1 and VL, respectively) of an intact antibody generally have similar structures. A single VH or VL domain may be sufficient to confer antigen-binding specificity. The constant regions of the antibodies may mediate the binding of the immunoglobulin to host tissues or factors, including various cells of the immune system (e.g. effector cells) and the first component (C1q) of the classical complement pathway. Furthermore native antibodies may belong to different classes. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁ IgG₂, IgG₃, IgG₄, IgGA₁ and IgGA₂. The heavy chain constant domains that correspond to the different classes of immunoglobulins are called α, δ, ε, γ, and μ, respectively.

The term “monoclonal antibody” as used herein refers to an antibody obtained from a population of substantially homogeneous antibodies, i.e., the individual antibodies comprising the population are identical and/or bind the same antigenic determinant (epitope), except for possible small amounts of variant antibodies, e.g., containing naturally occurring mutations or arising during production of a monoclonal antibody preparation. A monoclonal anti-HSP27 antibody preparation, for example, comprises a population of antibodies directed to a single of HSP27 antigenic determinant. Thus, the modifier “monoclonal” indicates the character of the antibody as being obtained from a substantially homogeneous population of antibodies. The modifier “monoclonal”, however, is not to be construed as requiring production of the antibody by any particular method.

The terms “polyclonal antibody preparations” or “polyclonal antibodies”, refer to different antibodies directed against different determinants (epitopes), as opposed to monoclonal antibody of a mono clonal antibody preparation is directed against a single determinant on an antigen. Thus, for example, a polyclonal anti-HSP27 antibody preparation comprises a population of antibodies directed to a plurality of HSP27 antigenic determinants. The modifier “polyclonal”, however, is not to be construed as requiring production of the antibodies by any particular method.

A “human antibody” is an antibody possessing an amino acid sequence which corresponds to that of an antibody produced by a human or a human cell or derived from a non-human source that utilizes human antibody repertoires or other human antibody-encoding sequences, and includes for example human native HSP27-antibodies. This definition of a human antibody specifically excludes a humanized antibody.

A “humanized antibody” refers to a chimeric antibody comprising amino acid residues from non-human and human sources. In certain embodiments, at least a portion of one, and more typically two, of the variable regions corresponds to a human antibody, and in which other portions of the antibody correspond to those of a non-human antibody. A humanized antibody optionally may comprise at least a portion of an antibody constant region derived from a human antibody.

The term “chimeric antibody” refers to an antibody in which a portion of the heavy and/or light chain is derived from a particular source or species, while the remainder of the heavy and/or light chain is derived from a different source or species.

The term “effective amount”, as used herein, refers to an amount of an active agent or pharmaceutical formulation, sufficient to induce a desired biological or therapeutic effect, including a prophylactic effect. Such effect can include an effect with respect to the signs, symptoms or causes of a disorder, or disease or any other desired alteration of a biological system. The effective amount can vary depending, for example, on the health condition, injury stage, disorder stage, or disease stage, weight, or sex of a subject being treated, timing of the administration, manner of the administration, age of the subject, and the like, all of which can be determined by those of skill in the art.

The terms “non-alcoholic fatty liver disease”, and the acronym “NAFLD”, as may be used interchangeably herein, refer to the accepted medical definition of non-alcoholic fatty liver disease, and includes a liver disease state in a subject, where excessive fat deposition, resulting in a fat content in liver cells of at least 5% can be observed, and wherein the subject does not have a history of sufficient alcohol consumption to evoke hepatic damage.

The terms “non-alcoholic steatohepatitis”, and the acronym “NASH”, as may be used interchangeably herein, refer to the accepted medical definition of non-alcoholic steatohepatitis and include, a liver disease state in a subject, where excessive fat deposition in liver cells can be observed, and where, furthermore, liver cell damage can be observed, including for example, in the form of liver fibrosis or liver cirrhosis, and wherein the subject does not have a history of sufficient alcohol consumption to evoke hepatic damage.

The term “pharmaceutically acceptable”, as used herein, refers to materials, including carriers, diluents, excipients or auxiliary agents, that are compatible with other materials in a pharmaceutical formulation and within the scope of reasonable medical judgement suitable for use in contact with a subject without excessive toxicity, allergic response, irritation, or other adverse response commensurate with a reasonable risk/benefit ratio.

The term “subject”, as used herein, refers to all members of the kingdom Animalia, and includes humans.

The terms “treating” and “treatment”, and the like, as used herein, are intended to mean obtaining a desirable physiological, pharmacological, or biological effect, and includes prophylactic and therapeutic treatment. The effect may result in the inhibition, attenuation, amelioration, or reversal of a sign, symptom or cause of a disorder, or disease, attributable to the disorder, or disease. Clinical evidence of the prevention or treatment may vary with the disorder, or disease, the subject and the selected treatment.

The term “pharmaceutical formulation”, as used herein, refers to a preparation in a form which allows an active ingredient contained therein to be effective, and which does not contain any other ingredients which cause excessive toxicity, an allergic response, irritation, or other adverse response commensurate with a reasonable risk/benefit ratio. The pharmaceutical formulation may contain other pharmaceutical ingredients such as carriers, diluents, excipients or auxiliary agents.

The term “chimeric”, as used herein in the context of nucleic acids, refers to at least two linked nucleic acids which are not naturally linked. Chimeric nucleic acids include linked nucleic acids of different natural origins. For example, a nucleic acid constituting a microbial promoter linked to a nucleic acid encoding a plant polypeptide is considered chimeric. Chimeric nucleic acids also may comprise nucleic acids of the same natural origin, provided they are not naturally linked. For example a nucleic acid constituting a promoter obtained from a particular cell-type may be linked to a nucleic acid encoding a polypeptide obtained from that same cell-type, but not normally linked to the nucleic acid constituting the promoter. Chimeric nucleic acids also include nucleic acids comprising any naturally occurring nucleic acids linked to any non-naturally occurring nucleic acids.

The terms “substantially pure” and “isolated”, as may be used interchangeably herein, describe a compound, e.g., a polypeptide or antibody, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides or antibodies, by chromatography, gel electrophoresis or HPLC analysis.

### General Implementation

As hereinbefore mentioned, the present disclosure relates to liver disorders and methods for treating liver disorders. In general, the herein provided methods can be used to treat non-alcoholic fatty liver disease (NAFLD), including non-alcoholic steatohepatitis (NASH). The inventor has discovered that, surprisingly, an HSP27 polypeptide, an anti-HSP27 antibody, or a mixture thereof, may be used to treat NAFLD. Since currently no pharmaceutical therapies for NAFLD currently exist, the methods of the present disclosure are desirable. In one aspect, the methods of the present disclosure represent a pharmaceutical therapeutic option that can alleviate symptoms in patients diagnosed with NAFLD or NASH.

By way of brief background, HSP27 is a member of a class of proteins known as small heat shock proteins (sHSP's). In vivo HSP27 is primarily involved in providing thermoprotection and support survival under cellular stress conditions. By acting as a chaperone protein HSP27 is believed to inhibit protein aggregation and stabilize partially denatured proteins. A further function of HSP27 includes binding of cytochrome c to thereby prevent the activation of caspases, proteins known to play a role in programmed cell death (apoptosis). Thus, HSP27 can function as an anti-apoptotic protein. For a more detailed review of HSP27, see, for example: Bakthisaran, R. et al., 2015, Biochimica et Biophysica Acta 1854: 291-319; and Butalan, Z et al., 2016, Front. Immunol. 7:285.

In accordance with one aspect hereof, the present disclosure provides, in at least one embodiment, a method for treating non-alcoholic fatty liver disease, the method comprising administering to a human subject in need thereof a pharmaceutical formulation comprising at least one of (i) an HSP27 polypeptide or an immunologically equivalent portion thereof, or (ii) an anti-HSP27 antibody or a functional antibody fragment thereof, wherein the pharmaceutical formulation is administered in an effective amount to treat non-alcoholic fatty liver disease in the subject.

In another aspect, the present disclosure further provides, in at least one embodiment, a use of at least one of (i) a HSP27 polypeptide or an immunologically equivalent portion thereof, or (ii) an anti-HSP27 antibody or functional antibody fragment thereof in the manufacture of a medicament for the treatment of non-alcoholic fatty liver disease in a human subject.

In another aspect, the present disclosure provides, in at least one embodiment, a use of a pharmaceutical formulation comprising at least one of (i) a HSP27 polypeptide or an immunologically equivalent portion thereof, or (ii) an anti-HSP27 antibody or functional antibody fragment thereof, to treat non-alcoholic fatty liver disease in a human subject.

In another aspect, the present disclosure provides, in at least one embodiment, a pharmaceutical formulation comprising at least one of (i) a HSP27 polypeptide or an immunologically equivalent portion thereof, or (ii) an anti-HSP27 antibody or functional antibody fragment thereof, for use in treating non-alcoholic fatty liver disease in a human subject in need thereof.

In general, according to an aspect, in to order to practice the methods and uses of the present disclosure, initially pharmaceutical formulations comprising (i) HSP27 polypeptide or an immunologically equivalent portion thereof, or (ii) an anti-HSP27 antibody or a functional HSP27 antibody fragment, or (iii) a mixture comprising both an HSP27 polypeptide or an immunologically equivalent portion thereof and an anti-HSP27 antibody or a functional HSP27 antibody fragment, are prepared or obtained. These formulations are administered in effective amounts to a subject in need thereof to treat non-alcoholic fatty liver disease. Thus next, suitable preparations comprising an HSP27 polypeptide or an immunologically equivalent portion thereof, or an anti-HSP27 antibody, or a functional HSP27 antibody fragment, or mixtures thereof, will be described. Thereafter pharmaceutical formulations comprising an HSP27 polypeptide or an immunologically equivalent portion thereof, or an anti-HSP27 antibody, or a functional HSP27 antibody fragment thereof, or mixtures thereof, and methods of preparing pharmaceutical formulations and administering the same to a subject in need thereof will be described.

Thus, initially, considering preparations of HSP27 polypeptide or an immunologically equivalent portion thereof, in an aspect, preparations containing an HSP27 polypeptide or an immunologically equivalent portion thereof can be prepared biosynthetically using a host cell system. In this respect, an isolated nucleic acid encoding an amino acid sequence corresponding with an HSP27 polypeptide or an immunologically equivalent portion thereof can be introduced in host cells and expressed therein.

According to an aspect, in an example embodiment, a nucleic acid sequence encoding an HSP27 polypeptide may be selected, wherein such nucleic acid includes SEQ.ID NO: 1 set forth herein. A selected example HSP27 polypeptide includes SEQ.ID NO: 2, which is a polypeptide encoded by SEQ.ID NO: 1.

Furthermore, according to an aspect, in an example embodiment, a nucleic acid sequence encoding an immunologically equivalent portion of an HSP27 polypeptide may be selected. Example nucleic acids encoding an immunologically equivalent portion of HSP27 polypeptide include SEQ.ID NO: 15, SEQ.ID NO: 16, SEQ.ID NO: 17, SEQ.ID NO: 18, SEQ.ID NO: 19, SEQ.ID NO: 20, SEQ.ID NO: 21, SEQ.ID NO: 22, and SEQ.ID NO: 23 set forth herein. Selected example immunologically equivalent portions of HSP27 polypeptides include SEQ.ID NO: 6, SEQ.ID NO: 7, SEQ.ID NO: 8, SEQ.ID NO: 9, SEQ.ID NO: 10, SEQ.ID NO: 11, SEQ.ID NO: 12, SEQ.ID NO: 13, and SEQ.ID NO: 14.

According to an aspect, further example nucleic acid sequences encoding HSP 27 polypeptides or an immunologically equivalent portion thereof that can be selected and used in accordance herewith include a nucleic acid sequence selected from:
(a) a nucleic acid sequence that is substantially identical to SEQ.ID NO: 1; SEQ.ID NO: 15, SEQ.ID NO: 16, SEQ.ID NO: 17, SEQ.ID NO: 18, SEQ.ID NO: 19, SEQ.ID NO: 20, SEQ.ID NO: 21, SEQ.ID NO: 22, and SEQ.ID NO: 23;
(b) a nucleic acid sequence that is substantially identical to SEQ.ID NO: 1; SEQ.ID NO: 15, SEQ.ID NO: 16, SEQ.ID NO: 17, SEQ.ID NO: 18, SEQ.ID NO: 19, SEQ.ID NO: 20, SEQ.ID NO: 21, SEQ.ID NO: 22, and SEQ.ID NO: 23, but for the degeneration of the genetic code;
(c) a nucleic acid sequence that is complementary to SEQ.ID NO: 1; SEQ.ID NO: 15, SEQ.ID NO: 16, SEQ.ID NO: 17, SEQ.ID NO: 18, SEQ.ID NO: 19, SEQ.ID NO: 20, SEQ.ID NO: 21, SEQ.ID NO: 22, and SEQ.ID NO: 23;
(d) a nucleic acid sequence encoding a polypeptide having the amino acid sequence set forth in SEQ.ID NO: 2, SEQ.ID NO: 6, SEQ.ID NO: 7, SEQ.ID NO: 8, SEQ.ID NO: 9, SEQ.ID NO: 10, SEQ.ID NO: 11, SEQ.ID NO: 12, SEQ.ID NO: 13, and SEQ.ID NO: 14;
(e) a nucleic acid sequence that encodes an immunologically equivalent functional variant of the amino acid sequence set forth in SEQ.ID NO: 2, SEQ.ID NO: 6, SEQ.ID NO: 7, SEQ.ID NO: 8, SEQ.ID NO: 9, SEQ.ID NO: 10, SEQ.ID NO: 11, SEQ.ID NO: 12, SEQ.ID NO: 13, and SEQ.ID NO: 14; and
(f) a nucleic acid sequence that hybridizes under stringent conditions to any one of the nucleic acid sequences set forth in (a), (b), (c), (d), or (e).

According to an aspect, nucleic acid sequences encoding HSP27 polypeptides that can be selected and used in accordance herewith include nucleic acid sequences encoding portions of HSP27 polypeptides, notably, in particular, HSP27 polypeptide portions which are immunologically equivalent to full length HSP27 polypeptides, including the polypeptide set forth in SEQ.ID NO: 2. Thus, further example nucleic acid sequences that may be used in selected embodiments include nucleic acid sequences encoding an amino acid sequence which is immunologically equivalent to an HSP27 polypeptide, the amino acid sequence corresponding with a portion, including, preferably, at least 10%, at least 20%, at least 30%, at least 40%, at least 50%, at least 60%, at least 70%, at least 80, at least 90% or at least 95%, of a full length HSP27 polypeptide, including the HSP27 polypeptide set forth in SEQ.ID NO: 2, or 10 consecutive amino acids and up to 150 amino acids, including of 10, 20, 30, 40, 50, 60, 70, 80, 90, 100, 110, 120, 130, 140, or 150 of a full length HSP27 polypeptide, including the HSP27 polypeptide set forth in SEQ.ID NO: 2.

Furthermore, in an aspect, in one embodiment, an immunologically equivalent portion of an HSP27 polypeptide that may be used in accordance herewith includes a portion of HSP27 (i.e. SEQ.ID NO: 2) including at least 30, at least 40, at least 50, at least 60, at least 70, at least 80, at least 90, at least 100, or at least 105 consecutive amino acids of the 105 N-terminal amino acid residue portion of HSP27. For clarity, the 105 N-terminal amino acid residue portion of HSP27 is set forth herein separately as SEQ.ID NO: 43, encoded by the nucleic acid sequence set forth SEQ.ID NO: 44.

Furthermore, in an aspect, in one embodiment, an immunologically equivalent portion of an HSP27 polypeptide that may be used in accordance herewith includes a HSP27 polypeptide portion comprising or consisting of at least one, at least two, at least three, at least four, at least five, at least six, at least seven, at least eight, or all nine, of the HSP27 epitopes set forth in SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14.

Furthermore, in aspect, in one embodiment, an immunologically equivalent portion of an HSP27 polypeptide that may be used in accordance herewith includes a portion of HSP27 including at least 30, at least 40, at least 50, at least 60, at least 70, at least 80, at least 90, at least 100, or at least 105 consecutive amino acids of the 105 N-terminal amino acid residue portion of SEQ.ID NO: 2.

Furthermore, in aspect, in one embodiment, an immunologically equivalent portion of the HSP27 polypeptide that may be used is a polypeptide comprising at least two of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, and at least 30 consecutive amino acids of the 105 N-terminal amino acid residue portion of SEQ.ID NO: 2.

Furthermore, in an aspect, in one embodiment, an immunologically equivalent portion of the HSP27 polypeptide that may be used is a polypeptide comprising at least three of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, and at least 50 consecutive amino acids of the 105 N-terminal amino acid residue portion of SEQ.ID NO: 2.

Furthermore, in an aspect, in one embodiment, an immunologically equivalent portion of the HSP27 polypeptide that may be used is a polypeptide comprising at least four of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, and at least 75 consecutive amino acids of the 105 N-terminal amino acid residue portion of SEQ.ID NO: 2.

Furthermore, in an aspect, in an embodiment, immunologically equivalent portions of an HSP27 polypeptide that may be used include a polypeptide comprising or consisting of SEQ.ID NO: 25 (comprising the HSP27 epitopes represented by SEQ.ID NO: 6 and SEQ.ID NO: 7), SEQ.ID NO: 26 (comprising the HSP27 epitopes represented by SEQ.ID NO: 6, SEQ.ID NO: 7, and SEQ.ID NO: 8), SEQ.ID NO: 27 (comprising the HSP27 epitopes represented by SEQ.ID NO: 6, SEQ.ID NO: 7, SEQ.ID NO: 8, and SEQ.ID NO: 9), SEQ.ID NO: 28 (comprising the Hsp27 epitopes represented by SEQ.ID NO: 6, SEQ.ID NO: 7, SEQ.ID NO: 8, SEQ.ID NO: 9, and SEQ.ID NO: 10), SEQ.ID NO: 29 (comprising the HSP27 epitopes represented by SEQ.ID NO: 6, SEQ.ID NO: 7, SEQ.ID NO: 8, SEQ.ID NO: 9, SEQ.ID NO: 10, and SEQ.ID NO: 11), SEQ.ID NO: 30 (comprising the HSP27 epitopes represented by SEQ.ID NO: 6, SEQ.ID NO: 7, SEQ.ID NO: 8, SEQ.ID NO: 9, SEQ.ID NO: 10, SEQ.ID NO: 11, and SEQ.ID NO: 12), SEQ.ID NO: 31 (comprising the HSP27 epitopes represented by SEQ.ID NO: 6, SEQ.ID NO: 7, SEQ.ID NO: 8, SEQ.ID NO: 9, SEQ.ID NO: 10, SEQ.ID NO: 11, SEQ.ID NO: 12, and SEQ.ID NO: 13), SEQ.ID NO: 32 (comprising the HSP27 epitopes represented by SEQ.ID NO: 6, SEQ.ID NO: 7, SEQ.ID NO: 8, SEQ.ID NO: 9, SEQ.ID NO: 10, SEQ.ID NO: 11, SEQ.ID NO: 12, SEQ.ID NO: 13, and SEQ.ID NO: 14), and SEQ.ID NO: 33 (comprising the HSP27 epitopes represented by SEQ.ID NO: 8, SEQ.ID NO: 9, SEQ.ID NO: 10, SEQ.ID NO: 11, and SEQ.ID NO: 12).

Furthermore, in an aspect, in an embodiment, immunologically equivalent portions of an HSP27 polypeptide that may be used include a polypeptide encoded by any one of the nucleic acid sequences set forth in SEQ.ID NO: 34; SEQ.ID NO: 35; SEQ.ID NO: 36; SEQ.ID NO: 37; SEQ.ID NO: 38; SEQ.ID NO: 39; SEQ.ID NO: 40; SEQ.ID NO: 41; and SEQ.ID NO: 42.

As is known to those of skill in the art, expression of nucleic acids in a host cell, to thereby biosynthetically produce a protein, can be achieved by providing one or more nucleic acids capable of controlling expression in a host cell, and operably linking the one or more nucleic acids capable of controlling expression in a host cell to the nucleic acid one wishes to express. Such operable linking of a nucleic acid controlling expression generally involves linking in the 5′ to 3′ direction of expression the nucleic acid capable of controlling expression in a host cell to the nucleic acid one wishes to express. Thus, within the context of the instant disclosure, a nucleic acid encoding an HSP27 polypeptide, including, for example SEQ.ID NO: 1 or SEQ.ID NO: 3, or immunologically equivalent portions thereof, including portions comprising one or more of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, can be linked to a nucleic acid controlling expression in a host cell. Suitable nucleic acid sequences capable of controlling expression in host cells that may be used herein include any transcriptional promoter capable of controlling expression of polypeptides in host cells. Generally, promoters obtained from bacterial cells are used when a bacterial host cell is selected, while a yeast promoter will be used when a yeast host cell is selected, an animal cell promoter will be used when an animal cell is selected, and so on. The obtained nucleic acid comprising a promoter and the nucleic acid expressing an HSP27 polypeptide or immunologically equivalent portion thereof is generally a chimeric nucleic acid. Further nucleic acid elements capable elements of controlling expression in a host cell include transcriptional terminators, enhancers and the like, all of which may be included in the chimeric nucleic acid sequences of the present disclosure.

In accordance with an aspect of the present disclosure, the chimeric nucleic acid sequences including a nucleic acid sequence expressing an HSP27 polypeptide or an immunologically equivalent portion thereof can be integrated into a recombinant expression vector which ensures good expression in the host cell, wherein the recombinant expression vector is suitable for expression in a host cell. The term “suitable for expression in a host cell” means that the recombinant expression vector comprises the chimeric nucleic acid linked to genetic elements required to achieve expression in a cell. As noted, such genetic elements can include transcriptional promoters, terminators and enhancers, and the like. Further genetic elements that may be included in the expression vector are one or more nucleic acid sequences encoding marker genes, and one or more origins of replication. In some embodiments, the expression vector can freely replicate in the host cell. In other embodiments, the chimeric nucleic acid can be integrated into the host cell's genomic DNA. In some embodiments, the expression vector further can comprise genetic elements required for the integration of the vector or a portion thereof in the host cell's genome.

Marker genes that may be used in accordance with the present disclosure include all genes that allow the distinction of transformed cells from non-transformed cells, including all selectable and screenable marker genes. A marker gene may be a resistance marker such as an antibiotic resistance marker against, for example, kanamycin, chloramphenicol, methotrexate, or ampicillin. In other instances, a marker gene may be a gene which allows a cell to produce an essential nutrients, for example amino acids.

Turning now to the host cell, it is noted, initially, that any host cell which upon cultivation expresses the nucleic acid sequence encoding a HSP27 polypeptide or an immunologically equivalent portion thereof can be selected and used. Suitable host cells in this respect include, for example, microbial cells, such as bacterial cells, yeast cells, for example, and algal cells or animal cells. A variety of techniques and methodologies to manipulate host cells to introduce nucleic acid sequences in cells and attain expression exists and are well known to the skilled artisan. These methods include, for example, cation based methods, for example, lithium ion or calcium ion based methods, electroporation, biolistics, and glass beads based methods. As will be known to those of skill in the art, depending on the host cell selected, the methodology to introduce nucleic acid material in the host cell may vary, and, furthermore, methodologies may be optimized for uptake of nucleic acid material by the host cell, for example, by comparing uptake of nucleic acid material using different conditions. Detailed guidance can be found, for example, in Sambrook et al., Molecular Cloning, a Laboratory Manual, Cold Spring Harbor Laboratory Press, 2012, Fourth Ed. It is noted that the chimeric nucleic acid is a non-naturally occurring chimeric nucleic acid sequence and can be said to be heterologous to the host cell.

One example host cell that conveniently may be used is Escherichia coli. The preparation of the E. coli vectors may be accomplished using commonly known techniques such as restriction digestion, ligation, gel electrophoresis, DNA sequencing, the polymerase chain reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Suitable promoter sequences for use in E. coli include, for example, the T7 promoter, the T5 promoter, tryptophan (trp) promoter, lactose (lac) promoter, tryptophan/lactose (tac) promoter, lipoprotein (lpp) promoter, and λ phage PL promoter. Typically, cloning vectors contain a marker, for example, an antibiotic resistance marker, such as ampicillin or kanamycin resistance marker, allowing selection of transformed cells. Nucleic acid sequences may be introduced in these vectors, and the vectors may be introduced in E. coli by preparing competent cells, electroporation or using other well-known methodologies to a person of skill in the art. E. coli may be grown in an appropriate medium, such as Luria-Broth medium and harvested. Recombinant expression vectors may readily be recovered from cells upon harvesting and lysing of the cells.

Another example host cell that may be conveniently used is a yeast cell. Example yeast host cells that can be used are yeast cells belonging to the genus Candida, Kluyveromyces, Saccharomyces, Schizosaccharomyces, Pichia, Hansenula, and Yarrowia. In specific example embodiments, the yeast cell can be a Saccharomyces cerevisiae cell, a Yarrowia lipolytica cell, or Pichia pastoris cell.

A number of vectors exist for the expression of recombinant proteins in yeast host cells. Examples of vectors that may be used in yeast host cells include, for example, Yip type vectors, YEp type vectors, YRp type vectors, YCp type vectors, pGPD-2, pAO815, pGAPZ, pGAPZα, pHIL-D2, pHIL-S1, pPIC3.5K, pPIC9K, pPICZ, pPICZα, pPIC3K, pHWO10, pPUZZLE and 2 μm plasmids. Such vectors are known to the art and are, for example, described in Cregg et al., Mol Biotechnol. (2000) 16(1): 23-52. Suitable promoter sequences for use in yeast host cells are also known and described, for example, in Mattanovich et al., Methods Mol. Biol., 2012, 824:329-58, and in Romanos et al., 1992, Yeast 8: 423-488. Examples of suitable promoters for use in yeast host cells include promoters of glycolytic enzymes, like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPGI), the 3-phosphoglycerate kinase promoter (PPGK), the glycerol aldehyde phosphate dehydrogenase promoter (PGAP), translation elongation factor promoter (PTEF), S. cerevisiae enolase (ENO-1), S. cerevisiae galactokinase (GAL1), S. cerevisiae alcohol dehydrogenase/glyceraldehyde-3-phosphate dehydrogenase (ADH1, ADH2/GAP), S. cerevisiae triose phosphate isomerase (TPI), S. cerevisiae metallothionein (CUP1), and S. cerevisiae 3-phosphoglycerate kinase (PGK), and the maltase gene promoter (MAL). Marker genes suitable for use in yeast host cells are also known to the art. Thus, antibiotic resistance markers, such as ampicillin resistance markers, can be used in yeast, as well as marker genes providing genetic functions for essential nutrients, for example, leucine (LEU2), tryptophan (TRP1 and TRP2), uracil (URA3, URA5, URA6), histidine (HIS3), and the like. Methods for introducing vectors into yeast host cells can, for example, be found in S. Kawai et al., 2010, Bioeng. Bugs 1(6): 395-403.

A further example of host cells that may be used in accordance herewith are animal host cells. These include, for example, mammalian cells, such as Chinese Hamster Ovary cells (CHO) cells, or lymphoid cells (e.g. Y0, NS0, or Sp20 cells), which are able to grow and survive when placed in either monolayer culture or suspension culture in medium containing appropriate nutrients and/or growth factors. Examples of expression vectors suitable for expression in animal cells include, but are not limited to, BPV-1, pHyg, pRSV, pIRES (Clontech), and pSG5 vectors (Stratagene). Selectable markers can used in to confer resistance to the cells harboring the vector to allow their selection in appropriate selection medium. A number of selection systems can be used, including but not limited to, the Herpes Simplex Virus thymidine kinase (HSV TK), (Wigler et al., 1977, Cell, 11:223), hypoxanthine-guanine phosphoribosyltransferase (HGPRT), (Szybalska and Szybalski, 1992, Proc. Natl. Acad. Sci. USA, 48:202), and adenine phosphoribosyltransferase (Lowy et al., 1980, Cell, 22:817) genes. Further vectors, media and growth conditions for animal cells can be found in Sambrook et al., Molecular Cloning, a Laboratory Manual, Cold Spring Harbor Laboratory Press, 2012, Fourth Ed.

Yet other example host cells that may be used in accordance herewith are plant cells. Methods for introducing nucleic acids in plant cells are known to those of skill in the art. Agrobacterium mediated plant cell transformation methods are described, for example, by Gelvin S. in Microbiol. Mol. Biol. Rev., 2003, 67(1): 16-37, and physical transformation based methods for plant cells are described by Rivera A. L. et al., 2012, Phys. Life Rev. 9(3): 308-345. Plant selectable marker genes are known to those of skill in the art and include antibiotic resistance genes, for example kanamycin resistance genes, and herbicide resistance genes, such as the bar and pat genes (Wohlleben et al., 1988, Gene 70:25-37). Screenable markers that may be employed to identify plant transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403). Plant promoters are also known to those in the art and include, for example, constitutive promoters, such as the 35S cauliflower mosaic virus (CaMV) promoter (Rothstein et al., 1987, Gene 53: 153-161), the rice actin promoter (McElroy et al., 1990, Plant Cell 2:163-171; U.S. Pat. No. 6,429,357), a ubiquitin promoter, such as the corn ubiquitin promoter (U.S. Pat. Nos. 5,879,903 and 5,273,894), and the parsley ubiquitin promoter (Kawalleck, P. et al., 1993, Plant Mol. Biol. 21:673-684), and organ specific promoters, such as seed specific promoters, for example, a phaseolin promoter (Sengupta-Gopalan et al., 1985, Proc. Natl. Acad. Sci. USA 82: 3320-3324), or an oleosin promoter (U.S. Pat. No. 5,792,922).

Further, guidance with respect to the preparation of expression vectors and introduction thereof into host cells, may be found in, for example: Sambrook et al., Molecular Cloning, a Laboratory Manual, Cold Spring Harbor Laboratory Press, 2012, Fourth Ed.

Thus, to briefly recap, a host cell comprising a chimeric nucleic acid comprising (i) a nucleic acid sequence encoding an HSP27 polypeptide, including, SEQ.ID NO: 1, for example, or an immunologically equivalent portion thereof, SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, for example; and (ii) a nucleic acid sequence capable of controlling expression of the nucleic acid sequence encoding an HSP27 polypeptide or an immunologically equivalent portion thereof in a host cell can be prepared in accordance with the present disclosure.

In accordance herewith, host cells are grown to multiply and to express a chimeric nucleic acid. Expression of the chimeric nucleic acid results in the biosynthetic production in the host cell of an HSP27 polypeptide or an immunologically equivalent portion thereof. Growth media and growth conditions can vary depending on the host cell that is selected, as will be readily appreciated to those of ordinary skill in the art. Growth media typically contain a carbon source, one or several nitrogen sources, essential salts including salts of potassium, sodium, magnesium, phosphate and sulphate, trace metals, water soluble vitamins, and process aids including but not limited to antifoam agents, protease inhibitors, stabilizers, ligands and inducers. Typical carbon sources are e.g. mono- or disaccharides. Typical nitrogen sources are, e.g. ammonia, urea, amino acids, yeast extract, corn steep liquor and fully or partially hydrolyzed proteins. Typical trace metals are e.g. Fe, Zn, Mn, Cu, Mo and H₃BO₃. Typical water soluble vitamins are e.g. biotin, pantothenate, niacin, thiamine, p-aminobenzoic acid, choline, pyridoxine, folic acid, riboflavin and ascorbic acid. Further, specific example media include liquid culture media for the growth of yeast cells and bacterial cells including, Luria-Bertani (LB) broth for bacterial cell cultivation, and yeast extract peptone dextrose (YEPD or YPD), for yeast cell cultivation, and CD-CHO medium, or Ham's F10 medium for growing CHO cells. Further media and growth conditions can be found in Sambrook et al., Molecular Cloning, a Laboratory Manual, Cold Spring Harbor Laboratory Press, 2012, Fourth Ed.

Upon production by the host cells of an HSP27 polypeptide or an immunologically equivalent portion thereof, the HSP27 polypeptide or immunologically equivalent portion thereof may be recovered from the host cells, and separated from other constituents, such as cellular debris, or media constituents, for example. Separation techniques will be known to those of skill in the art and include a variety of different protein purification techniques including, e.g. ion-exchange chromatography, size exclusion chromatography, affinity chromatography, hydrophobic interaction chromatography, reverse phase chromatography, gel filtration, etc. Further general guidance with respect to protein purification may for example be found in: Cutler, P. Protein Purification Protocols, Humana Press, 2004, Second Ed. Thus substantially pure preparations of HSP27 polypeptides or immunologically equivalent portion thereof may be obtained. The recovered HSP27 polypeptide or immunologically equivalent portion thereof may be obtained in a more or less pure form, for example, a preparation of HSP27 polypeptides or immunologically equivalent portion thereof of at least about 60% (w/v), about 70% (w/v), about 80% (w/v), about 90% (w/v), about 95% (w/v), or about 99% (w/v) purity may be obtained.

It is noted that the cells in some embodiments may secrete a portion of the produced HSP27 polypeptide or immunologically equivalent portion thereof in the cell growth medium, thus a portion of the produced HSP27 polypeptide or immunologically equivalent portion thereof may be recovered from the cells and a further portion of the HSP27 polypeptide or immunologically equivalent portion thereof may be recovered from the growth medium.

Turning now to anti-HSP27 antibodies, or functional antibody fragments thereof, in general, it is noted that any anti-HSP27 antibody or anti-HSP27 functional antibody fragment may be selected and employed in accordance herewith. In what follows next various example anti-HSP27 antibodies and methods of making anti-HSP27 antibody preparations are described.

In accordance with one aspect, in an example embodiment, a polyclonal anti-HSP27 antibody preparation can be obtained and used. Anti-HSP27 polyclonal antibodies may be prepared by injecting an HSP27 polypeptide, which may be obtained as hereinbefore described, or an or an immunologically equivalent portion thereof, into an animal of choice, a mouse, a rabbit or a goat, for example. After a series of injections over a specific length of time, the animal will have created antibodies against the HSP27 protein. Blood can then be extracted from the animal, and used to obtain an anti-HSP27 antibody preparation. Such preparations contain intact native antibodies, including generally a preponderance of antibodies belonging to the IgG class. Techniques for producing polyclonal antibodies are generally known to the art, see for example: Leenaars M. et al., 2005, ILAR Journal, 46 (3) 269-279. An example method or preparing anti-HSP27 polyclonal antibodies is further described in Example 2.

In accordance with one aspect, in a further example embodiment, a monoclonal anti-HSP27 antibody preparation can be obtained and prepared. Anti-HSP27 monoclonal antibodies may be prepared using hybridoma-based methods. Human myeloma and mouse-human hetero myeloma cell lines for the production of monoclonal antibodies, for example, have been described, see: e.g., Kozbor, 1984, J. Immunol., 133:3001; Brodeur et al. Monoclonal Antibody Production Techniques and Applications, pp. 51-63 (Marcel Dekker, Inc, New York, 1987); and Boerner et al. (1991) J. Immunol., 147: 86. Antibodies generated via human B-cell hybridoma technology are also described in Li et al., 2006, Proc. Natl. Acad. Sci. USA, 103: 3557-3562. Monoclonal antibodies may also be generated by isolating Fv clone variable domain sequences selected from phage display libraries. Such variable domain sequences may then be combined with a desired constant domain. For example, a variety of methods are known in the art for generating phage display libraries, and screening such libraries for antibodies possessing the desired binding characteristics. Such methods are reviewed, e.g., in Hoogenboom et al., 2001, in Methods in Molecular Biology 178:1-37 (O'Brien et al., ed., Human Press, Totowa, N.J.).

In accordance with an aspect, in a further example embodiment, a human anti-HSP27 antibody preparation can be obtained and prepared. Human Anti-HSP27 antibodies may be prepared by administering an HSP27 polypeptide to a transgenic animal that has been modified to produce intact human antibodies or intact antibodies with human variable regions in response to an antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. Transgenic animal based techniques for preparing monoclonal antibodies are known to the art. For a review of methods for obtaining human antibodies from transgenic animals, see Lonberg, 2005, Nat. Biotech. 23:1117-1125. Furthermore human anti-HSP27 antibodies can also be prepared using hybridoma-based techniques and phage display techniques.

In accordance with an aspect, in a further example embodiment, a chimeric anti-HSP27 antibody preparation can be obtained and prepared. Chimeric anti-HSP27 antibodies include humanized anti-HSP27 antibodies. Typically, humanized anti-HSP27 antibodies are used to reduce the immunogenicity to humans while retaining the specificity with respect to the ability of the antibody to bind HSP27 polypeptide. Humanized antibodies and methods of making them are reviewed, e.g., in Almagro and Fransson, 2008, Front. Biosci. 13:1619-1633.

In different embodiments, anti-HSP27 monoclonal antibodies belonging to different classes may be prepared, for example monoclonal antibodies belong to the IgG class, the IgM class etc.

In accordance with an aspect, in a further example embodiment, variant anti-HSP27 antibody preparation can be obtained and prepared. Variant anti-HSP27 antibodies may be prepared by modifying the amino acid sequences of the antibodies. For example, it may be desirable to improve the binding affinity and/or other biological properties of the antibody. Amino acid sequence variants of an antibody may be prepared by introducing appropriate modifications into the nucleic sequence encoding the antibody, or by peptide synthesis. Such modifications include, for example, deletions from, and/or extensions to, and/or insertions into, and/or substitutions of residues within the amino acid sequences of the antibody. Any combination of deletion, extension, insertion, or substitution can be made to arrive at a final nucleic acid sequence construct, provided however, that final construct an HSP27 antibody or functional fragment thereof, is capable of binding HSP27 polypeptide or an immunologically equivalent portion thereof. In certain embodiments of variant anti-HSP27 antibodies included herein, one or more amino acid modifications may be introduced into the Fc region of an anti-HSP27 antibody, thereby generating an Fc region variant anti-HSP27 body. The Fc region variant may comprise a human Fc region sequence (e.g., a human IgG1, IgG2, IgG3 or IgG4 Fc region) comprising an amino acid modification (e.g. a substitution) at one or more amino acid positions. In certain embodiments, an anti-HSP27 antibody variant possesses some but not all effector functions, which make it a desirable candidate for applications in which the half-life of the antibody in vivo is important, yet certain effector functions, such as complement and antibody-dependent cell mediated cytotoxicity, for example are unnecessary or deleterious.

Further specific antibody variants include glycosylation variants. Native antibodies produced by mammalian cells typically comprise a branched, biantennary oligosaccharide that is generally attached by an N-linkage to Asn297 of the CH2 domain of the Fc region (i.e. the asparagine amino acid residue located at approximately residue 297 in the Fc region). See, e.g., Wright et al., 1997, MBTECH, 15:26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-HSP27 antibody of the present disclosure may be made in order to create antibody variants with certain improved properties. In one embodiment, for example, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. Such an antibody may exhibit antibody-dependent cell mediated cytotoxicity (ADCC).

Extensions to prepare antibody variants include amino-terminal or carboxy terminal extensions, ranging in length from, for example, one residue to a hundred or more residues. Extensions include extensions to increase the serum halve life of an antibody.

Further antibody variants include cysteine engineered antibody variants, in which one or more antibody residues are replaced with cysteine residues. By substituting certain residues with cysteine residues, reactive thiol groups are thereby introduced. These reactive thiol groups may be used to conjugate the antibody to other moieties, such as a cytotoxin or a drug e.g., an immunosuppressant.

In accordance with an aspect, in yet a further example embodiment, an anti-HSP27 functional fragment preparation can be obtained and prepared. Anti-HSP27 functional fragments include Fab, Fab′, Fab′-SH, F(ab′)2, Fv, and scFv fragments, and other fragments described below. For a review of certain antibody fragments, see Hudson et al. Nat. Med., 2003, 9:129-134. For a review of scFv fragments, see, e.g., Pluckthün, in The Pharmacology of Monoclonal Antibodies, 1994, vol. 113, Rosenburg and Moore eds., (Springer-Verlag, New York), pp. 269-315 (1994). For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, Hudson et al., Nat. Med., 2003, 9: 129-134, and Hollinger et al., Proc. Natl. Acad. Sci. (USA), 1993, 90: 6444-6448. Triabodies and tetrabodies are also described in Hudson et al., 1993, Nat. Med. 9: 129-134. Single-domain antibodies are antibody fragments comprising all or a portion of the heavy chain variable domain or all or a portion of the light chain variable domain of an antibody. In certain embodiments, a single-domain antibody is a human single-domain antibody (See, e.g., U.S. Pat. No. 6,248,516). Antibody fragments can be made by various techniques, including but not limited to proteolytic digestion of an intact antibody, as well as production by recombinant host cells (e.g. E. coli or phage), as described herein.

In accordance with an aspect, in a yet further example embodiment, an anti-HSP27 derivative preparation can be obtained and prepared. In certain embodiments, an anti-HSP27 antibody provided herein may be further modified to contain additional non-proteinaceous moieties that are known in the art and readily available. The moieties suitable for derivatization of the antibody include, but are not limited to, water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols (e.g. glycerol), polyvinyl alcohol, and mixtures thereof. The foregoing may have advantages in manufacturing due to an increase stability in aqueous solutions.

In an aspect, anti-HSP27 antibodies, or functional antibody fragments thereof may be produced using biosynthetically, using substantially similar techniques as hereinbefore described with respect to HSP27 polypeptides. Thus, nucleic acids encoding an amino acid sequence comprising the VL and/or an amino acid sequence comprising the VH of the antibody (e.g. the light and/or heavy chains of the antibody) may be isolated and included in expression vectors. Thus, for example one may prepare: (1) an expression vector comprising a nucleic acid that encodes an amino acid sequence comprising the VL of the antibody and an amino acid sequence comprising the VH of the antibody, or (2) a first expression vector comprising a nucleic acid that encodes an amino acid sequence comprising the VL of the antibody and a second expression vector comprising a nucleic acid that encodes an amino acid sequence comprising the VH of the antibody. In one embodiment, the host cell can be e.g. a Chinese Hamster Ovary (CHO) cell or a lymphoid cell (e.g. Y0, NS0, or Sp20 cell). Furthermore, nucleic acids encoding an anti-hSP27 antibody may be expressed in bacterial cells, in particular when glycosylation and Fc effector function are not needed. For expression of antibody fragments and polypeptides in bacteria, see, e.g. U.S. Pat. Nos. 5,648,237, 5,789,199, and 5,840,523. Upon culturing of the cell, the anti-HSP27 antibody or functional antibody fragment thereof may be isolated from the bacterial cells and can be further purified, as desired, to obtain a preparation comprising anti-HSP27 antibodies or functional a functional fragment thereof.

In accordance herewith anti-HSP27 antibodies may also be obtained from a commercial supplier of anti-HSP27 antibodies. In this respect, polyclonal and monoclonal anti-HSP27 antibodies are offered for sale by numerous suppliers, including, for example, Abcam® plc (Cambridge, England), Thermo Fisher Scientific® Inc. (Waltham, Mass., USA), and Enzo Lifesciences® Inc. (Farmingdale, N.Y., USA). Furthermore, it is noted that anti-HSP27 antibodies have been known to the art since at least as early as 1992 (Santell, L., et al., 1992, Biochem J., 284: 705-710). Furthermore, anti-HSP27 antibodies have been available for commercial purchase since at least as early as 2005 (De Souza, A. I. et al., 2005, Circulation Research, 1-7).

Thus, to briefly recap, preparations of HSP27 polypeptides or an immunologically equivalent portion thereof, preparations of anti-HSP27 antibodies, and preparations of functional HSP27 antibody fragments, and methods of making or obtaining the foregoing have been described. Turning next to the preparation of pharmaceutical preparations, the present disclosure involves, in an aspect the preparation of a pharmaceutical formulation comprising (i) an HSP27 polypeptide or an immunologically equivalent portion thereof, or (ii) an anti-HSP27 antibody or a functional antibody fragment thereof, or (iii) a mixture of HSP27 polypeptide or an immunologically equivalent portion thereof and an anti-HSP27 antibody or a functional HSP27 antibody fragment.

In accordance with an aspect, in an example embodiment, a pharmaceutical formulation is prepared comprising an HSP27 polypeptide or an immunologically equivalent portion thereof, wherein the pharmaceutical formulation is free or substantially free of an anti-HSP27 antibody or a functional antibody fragment thereof.

In accordance with an aspect, in an example embodiment, a pharmaceutical formulation is prepared comprising an anti-HSP27 antibody or a functional fragment thereof, wherein the pharmaceutical formulation is free or substantially free of HSP27 polypeptide or an immunologically equivalent portion thereof.

In accordance with an aspect, in an example embodiment, a pharmaceutical formulation is prepared comprising an HSP27 polypeptide or an immunologically equivalent portion thereof and at least on one of (i) anti-HSP27 antibody or (ii) a functional HSP27 antibody fragment. It is noted that in these formulations the HSP27 polypeptide or immunologically equivalent portion thereof and the anti-HSP27 antibody or the functional HSP27 antibody fragment may bind and form a polypeptide/antibody complex. Thus, an HSP27 polypeptide or an immunologically equivalent portion thereof and an anti-HSP27 antibody may form a polypeptide/antibody complex (i.e. an HSP27/anti-HSP27 antibody complex, or an immunologically equivalent portion of an HSP27 polypeptide/anti-HSP27 antibody complex), or a HSP27 polypeptide or an immunologically equivalent portion thereof and an anti-HSP27 functional antibody fragment may form a polypeptide/antibody complex (i.e. an HSP27/anti-HSP27 functional antibody fragment complex or an immunologically equivalent portion of an HSP27 polypeptide/anti-HSP27 functional antibody fragment complex).

In order to prepare a pharmaceutical formulation, a preparation comprising (i) HSP27 polypeptide or an immunologically equivalent portion thereof, or (ii) an anti-HSP27 antibody or a functional antibody fragment thereof, or mixtures of (i) and (ii), may be combined with at least one other pharmaceutically acceptable ingredient, including but not limited to, a diluent, an excipient, a carrier, or mixtures thereof, whereby the HSP27 polypeptide or immunologically equivalent portion thereof, or the anti-HSP27 antibody or the functional antibody fragment thereof, or the mixtures thereof, and at least one pharmaceutically acceptable ingredient are mixed together or blended or homogenized or otherwise prepared until the pharmaceutical formulation is formed.

The amount of HSP27 polypeptide or immunologically equivalent portion thereof, or an anti-HSP27 antibody or a functional antibody fragment thereof, or mixtures thereof in the pharmaceutical formulation may vary. In general, consideration is given to the dose to be administered to a subject. Doses of HSP27 polypeptide or immunologically equivalent portion can range, for example, from a dose of about 25 μg to about 10 mg, preferably 100 μg to about 2.5 mg, and amounts therebetween, for human subjects, for example, a dose of about 100 μg, about 200 μg, about 300 μg, about 400 μg, about 500 μg, about 750 μg, about 1 mg, or about 2.5 mg. Doses of anti-HSP27 antibody, or a functional antibody fragment thereof, can range, for example, from a dose of about 100 mg to about 5 g, and amounts therebetween, for human subjects, for example, a dose of about 100 mg, about 250 mg, about 500 mg, about 1 g, about 1.5 g, about 2 g, about 2.5 g, about 3 g, about 3.5 g, about 4 g, about 4.5 g, or about 5 g. Furthermore, the amount of HSP27 polypeptide or an immunologically equivalent portion thereof, or an anti-HSP27 antibody or a functional antibody fragment thereof, or mixtures thereof in a dose will generally range from about 0.01% (w/v) to about ninety-nine percent (w/v) of the pharmaceutical formulation, preferably from about 0.1% (w/w) to about 70% (w/w), most preferably from about 1% (w/v) to about 30% (w/v) in combination with other pharmaceutically acceptable ingredients included in the formulation.

Concentrations of HSP27 polypeptide or an immunologically equivalent portion thereof, or an anti-HSP27 antibody or a functional antibody fragment thereof, or mixtures thereof, in the pharmaceutical formulation may vary. Thus, formulations comprising HSP27, or immunologically equivalent portions, may be prepared to contain HSP27 or an immunologically equivalent portion thereof in, for example, a concentration range of from about 25 μg/ml to about 50 mg/ml or concentrations therebetween, or from about 50 μg/ml to about 25 mg/ml or concentrations therebetween, or from about 100 μg/ml to about 10 mg/ml or concentrations therebetween, for example, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1 mg/ml, about 1.25 mg/ml, about 1.5 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 9 mg/ml, about 9.5 mg/ml, or about 10 mg/ml.

Formulations comprising an anti-HSP27 antibody or a functional antibody fragment thereof may be prepared to contain an anti-HSP27 antibody or a functional antibody fragment thereof in, for example, a concentration range from about 0.5 mg/ml to about 250 mg/ml, or concentrations therebetween, or from about 5 mg/ml to about 250 mg/ml, or concentrations therebetween, or from about 10 mg/ml to about 200 mg/ml, or concentrations therebetween, or from about 25 mg/ml to about 200 mg/ml, or concentrations therebetween, for example, about 25 mg/ml, about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, or about 200 mg/ml.

The concentration of HSP27 or an immunologically equivalent portion thereof, or anti-HSP27 antibody or a functional antibody fragment thereof, in the pharmaceutical formulation may be varied, depending on the dose to be administered to a recipient subject, which, as is understood by those of skill in art, may vary depending on the age and general condition of the recipient subject to be treated, the severity of the condition being treated, the particular preparation delivered, the site of administration, as well as other factors.

Pharmaceutical formulations comprising HSP27 polypeptide or an immunologically equivalent portion thereof, or an anti-HSP27 antibody or a functional antibody fragment thereof, or mixtures thereof, preferably further are prepared by combining HSP27 polypeptide or an immunologically equivalent portion thereof, or an anti-HSP27 antibody or a functional antibody fragment thereof, or mixtures thereof with e.g. a carrier, an excipient, a diluent and/or auxiliary substances, such as wetting or emulsifying agents, pH buffering substances and the like. These carriers, excipients, diluents and auxiliary substances are pharmaceutically acceptable ingredients. Pharmaceutically acceptable excipients include, but are not limited to, liquids such as water, saline, polyethylene glycol, hyaluronic acid, glycerol and ethanol. Pharmaceutically acceptable salts can also be included in the formulation, for example, mineral acid salts such as hydrochlorides, phosphates, sulfates, sodium salts, calcium salts, potassium salts, and the like; and the salts of organic acids such as acetates, propionates, benzoates, and the like. It is also preferred, although not required, that the pharmaceutical formulation will contain a pharmaceutically acceptable carrier that serves as a stabilizer, particularly in order to stabilize the polypeptides and antibodies or antibody fragments. Examples of suitable carriers that also act as stabilizers for peptides include, without limitation, pharmaceutical grades of dextrose, sucrose, lactose, sorbitol, inositol, dextran, and the like. Other suitable carriers include, again without limitation, starch, cellulose, sodium or calcium phosphates, citric acid, glycine, polyethylene glycols (PEGs), and combinations thereof. The selection and use of suitable excipients, carriers and diluents is taught in Gennaro, ed., Remington: The Science and Practice of Pharmacy, 20th Ed. (Lippincott Williams & Wilkins 2003), the disclosure of which is incorporated herein by reference.

In accordance with an aspect, in an example embodiment hereof wherein the pharmaceutical formulation comprises an HSP27 polypeptide or an immunologically equivalent portion thereof, the administration of the polypeptide to a subject may result in an immune response being elicited in the subject upon the administration thereof to the subject. The immune response may be a humoral immune response and include the production of native anti-HSP27 antibodies in the blood serum of the subject. Furthermore, the native anti-HSP27 antibodies may bind in vivo in the subject to form a polypeptide/antibody complex, i.e. a complex comprising HSP27 or an immunologically equivalent portion thereof and anti-HSP27 antibody. Such forming of a complex may involve binding of the native anti-HSP27 antibodies to the HSP27 polypeptide or immunologically equivalent portion thereof administered and/or to the subject's native HSP27 polypeptides.

In order to augment an immune response in a subject, formulations comprising HSP27 polypeptide or an immunologically equivalent portion thereof may further include adjuvants, such as pharmacological agents, cytokines, or the like. Suitable adjuvants include any substance that enhances the immune response of the subject to an HSP27 polypeptide or an immunologically equivalent portion thereof. Non-limiting examples of adjuvants include cytokines, e.g., IL-1, IL-2, IL-12, IL-6, and further include inorganic salts, e.g. aluminum hydroxide, aluminum phosphate, and calcium phosphate; oil emulsions, e.g. mineral oil, MF59, QS-21, Montamide ISA51 and ISA-720; Isocoms, e.g. ISCOMATRIX; microbial derivatives, e.g. MPLA, macrophage-activating protein-2, virosomes, LT/CT, CpG; natural polymers, e.g. polysaccharides; and synthetic polymers, e.g. polyanhydrides and polyesters.

In an aspect, the methods and uses of the present disclosure involve the administration to a subject of a pharmaceutical formulation comprising at least one of (i) an HSP27 polypeptide or an immunologically equivalent portion thereof, or (ii) an anti-HSP27 antibody or functional antibody fragment thereof. The pharmaceutical formulations may be administered intravenously, intramuscularly, subcutaneously, parenterally, spinally or epidermally (e.g., by injection or infusion). Depending on the route of administration, the HSP27 polypeptide or immunologically equivalent portion thereof or anti-HSP27 antibody, or functional antibody fragment thereof can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, e.g., intranasally, orally, vaginally, rectally, sublingually or topically.

In example embodiments, a pharmaceutical formulation comprising a concentration of HSP27 polypeptide, or an immunologically equivalent portion thereof, in a concentration of from about 100 μg/ml to about 1 mg/ml, or concentrations therebetween, for example, about 100 μg/ml, about 150 μg/ml, 200 μg/ml, about 250 μg/ml, 300 μg/ml, about 350 μg/ml, 400 μg/ml, about 450 μg/ml, 500 μg/ml, about 550 μg/ml, 600 μg/ml, about 650 μg/ml, 700 μg/ml, about 750 μg/ml, 800 μg/ml, about 850 μg/ml, 900 μg/ml, about 950 μg/ml, or about 1 mg/ml may be administered subcutaneously, by administration thereof to a human subject of a volume ranging, for example, from about 0.5 ml to about 2 ml.

In further example embodiments, a pharmaceutical formulation comprising a concentration of an anti-HSP27 antibody or functional antibody fragment thereof in a concentration of from about 25 mg/ml to about 100 mg/ml or concentrations therebetween, for example, about 25 mg/ml, about 30 mg/ml, 40 mg/ml, about 50 mg/ml, 60 mg/ml, about 70 mg/ml, 80 mg/ml, about 90 mg/ml, or about 100 mg/ml may be administered intravenously or subcutaneously, by administration thereof of to a human subject of a volume ranging, for example, from about 1 ml to about 500 ml.

As is well understood by those of skill in the art, the exact amount necessary, will vary depending on the age and general condition of the recipient subject to be treated, the severity of the condition being treated, the particular preparation delivered, the site of administration, as well as other factors. An appropriate therapeutically effective amount can be readily determined by one of skill in the art. Thus, a therapeutically effective amount of the present formulations will be an amount sufficient to bring about treatment of NAFLD or NASH, or to prevent NAFLD or NASH, and will fall in a relatively broad range that can be determined through routine trials.

In an aspect, the methods and uses of the present disclosure involve the administration to a subject of a pharmaceutical formulation comprising at least one of (i) an HSP27 polypeptide or an immunologically equivalent portion thereof, or (ii) an anti-HSP27 antibody or functional antibody fragment thereof. It is noted that depending on whether (i) a pharmaceutical formulation comprising an HSP27 polypeptide of immunologically equivalent portion thereof, and not anti-HSP27 antibody or functional antibody fragment is administered, or (ii) a pharmaceutical formulation comprising an anti-HSP27 antibody or functional antibody fragment, and not an HSP27 polypeptide of immunologically equivalent portion thereof is administered, or (iii) a pharmaceutical formulation comprising an anti-HSP27 antibody or functional antibody fragment, and an HSP27 polypeptide or immunologically equivalent portion thereof, is administered, the in vivo response of the subject in terms of binding of HSP27 polypeptide or immunologically equivalent portion thereof and anti-HSP27 antibody to thereby form a complex may vary. Thus, in an example embodiment, the pharmaceutical formulation can comprise an HSP27 polypeptide or and immunologically equivalent portion thereof, and not an anti-HSP27 antibody or a functional antibody fragment thereof. Upon administration, the HSP27 polypeptide or immunologically equivalent portion thereof can elicit an immune response in the subject and the production of native anti-HSP27 antibodies in the blood serum of the subject. Furthermore the HSP27 polypeptide or immunologically equivalent portion thereof and native anti-HSP27 antibodies can form a polypeptide/antibody complex in vivo in the subject, i.e. a HSP27 polypeptide/anti-HSP27 complex, or a HSP27 polypeptide portion immunologically equivalent to HSP27/anti-HSP27 antibody complex.

In another example embodiment, the pharmaceutical formulation can comprise an anti-HSP27 antibody or a functional fragment thereof, and not include an HSP27 polypeptide or immunologically equivalent portion thereof. Upon administration of the pharmaceutical formulation to a subject a complex can be formed between the anti-HSP27 antibody or a functional fragment thereof, and the native HSP27 polypeptide of the subject.

In another example embodiment, the pharmaceutical formulation can comprise an HSP27 polypeptide or an immunologically equivalent portion thereof and an anti-HSP27 antibody or anti-HSP27 functional antibody fragment thereof. The HSP27 or immunologically equivalent portion thereof and native anti-HSP27 antibodies can form a polypeptide/antibody complex or a polypeptide/anti-HSP27 functional antibody fragment complex. The complex being present in the formulation, as hereinbefore noted, or the complex can be formed in vivo in the subject upon administration.

As noted, the pharmaceutical formulations of the present disclosure may be used to treat NAFLD or NASH in a subject.

In some embodiments, the subject may exhibit and/or be diagnosed with hepatic steatosis.

In some embodiments, the subject may exhibit and/or be diagnosed with hepatic fibrosis.

In some embodiments, the subject may exhibit and/or be diagnosed with hepatic cirrhosis.

In an aspect, upon administration of a formulation comprising HSP27 or immunologically equivalent portion thereof, an anti-HSP27 antibody or an anti-HSP27 antibody fragment, or mixtures thereof to the subject, multiple biological reactions may occur in vivo in the subject. In an aspect, biological reactions reducing inflammatory signaling may occur in the subject. In particular, one or more of the following may be observed upon the administration to a subject: (i) in the in vivo presence of HSP27/anti-HSP27 antibody complex or HSP27/anti-HSP27 functional antibody fragment complex or immunologically equivalent HSP27 portion/anti-HSP27 antibody complex or immunologically equivalent HSP27 portion/anti-HSP27 functional antibody fragment complex, the binding of HSP27 or immunologically equivalent HSP27 portion to cell membranes of liver cells, including hepatocytes, Kupffer cells, hepatic lymphoid cells, hepatic dendritic cells, hepatic cholangiocytes, and hepatic stellate cells, or to cells in the circulatory system directed to the liver, such as macrophage cells, can be enhanced; (ii) in the in vivo presence of HSP27/anti-HSP27 antibody complex or HSP27/anti-HSP27 functional antibody fragment complex or immunologically equivalent HSP27 portion/anti-HSP27 antibody complex or immunologically equivalent HSP27 portion/anti-HSP27 functional antibody fragment complex, activation of NF-κB signaling by lipopolysaccharide through Toll Like Receptor 4 (TLR4) in liver cells, including hepatocytes, Kupffer cells, hepatic lymphoid cells, hepatic dendritic cells, hepatic cholangiocytes, and hepatic stellate cells, can be reduced and can effect an anti-inflammatory cytokine profile, notably a reduction in the production of the inflammatory cytokine IL-1p, and an increase in the production of the anti-inflammatory cytokine IL-10; (iii) in the in vivo presence of HSP27/anti-HSP27 antibody complex or HSP27/anti-HSP27 functional antibody fragment complex or immunologically equivalent HSP27 portion/anti-HSP27 antibody complex or immunologically equivalent HSP27 portion/anti-HSP27 functional antibody fragment complex, generally in the blood serum of the subject, the interaction of circulating oxidized low density lipoprotein (oxLDL) with subject's scavenger receptor SR-A1, notably liver cell SR-A1, can be reduced, and uptake of oxLDL by liver cells, including hepatocytes, Kupffer cells, hepatic lymphoid cells, hepatic dendritic cells, hepatic cholangiocytes, and hepatic stellate cells can be reduced; (iv) in the in vivo presence of HSP27/anti-HSP27 antibody complex or HSP27/anti-HSP27 functional antibody fragment complex or immunologically equivalent HSP27 portion/anti-HSP27 antibody complex or immunologically equivalent HSP27 portion/anti-HSP27 functional antibody fragment complex, generally in the blood serum of the subject, the interaction of circulating oxLDL with the subject's scavenger receptor CD-36, notably liver cell CD-36, can be reduced, and uptake of oxLDL by liver cells, including hepatocytes, Kupffer cells, hepatic lymphoid cells, hepatic dendritic cells, hepatic cholangiocytes, and hepatic stellate cells of oxidized low density lipoprotein (oxLDL) can be reduced; and (v) in the in vivo presence of HSP27/anti-HSP27 antibody complex or HSP27/anti-HSP27 functional antibody fragment complex or immunologically equivalent HSP27 portion/anti-HSP27 antibody complex or immunologically equivalent HSP27 portion/anti-HSP27 functional antibody fragment complex, HSP27 or immunologically equivalent HSP27 portion uptake by liver cells, including hepatocytes, Kupffer cells, hepatic lymphoid cells, hepatic dendritic cells, hepatic cholangiocytes, and hepatic stellate cells can be enhanced. Without wishing to be bound by theory, or mechanism, it is believed that one or more of the foregoing biological reactions can be therapeutically beneficial in the treatment of NAFLD or NASH in a subject.

As can now be understood, pharmaceutical formulations comprising at least one of (i) HSP27 polypeptide or immunologically equivalent portion thereof, or (ii) an anti-HSP27 antibody or a functional antibody fragment thereof, may be prepared and administered to a subject to treat NAFLD or NASH.

### SUMMARY OF SEQUENCES

SEQ.ID NO: 1 sets forth a polynucleotide sequence encoding a human HSP27 polypeptide.

SEQ.ID NO: 2 sets forth a human deduced amino acid sequence of a human HSP27 polypeptide.

SEQ.ID NO: 3 sets forth a human polynucleotide sequence encoding a HIS-tagged HSP27 polypeptide.

SEQ.ID NO: 4 sets forth a HIS-tagged deduced amino acid sequence of a human HSP27 polypeptide.

SEQ.ID NO: 5 sets forth a HIS-tagged human deduced amino acid sequence of an HSP27 polypeptide fragment referred herein as HSP27 rC1.

SEQ.ID NO: 6 sets forth a deduced amino acid sequence of a portion of a HSP27 polypeptide corresponding with a first HSP27 epitope.

SEQ.ID NO: 7 sets forth a deduced amino acid sequence of a portion of a HSP27 polypeptide corresponding with a second HSP27 epitope.

SEQ.ID NO: 8 sets forth a deduced amino acid sequence of a portion of a HSP27 polypeptide corresponding with a third HSP27 epitope.

SEQ.ID NO: 9 sets forth a deduced amino acid sequence of a portion of a HSP27 polypeptide corresponding with a fourth HSP27 epitope.

SEQ.ID NO: 10 sets forth a deduced amino acid sequence of a portion of a HSP27 polypeptide corresponding with a fifth HSP27 epitope.

SEQ.ID NO: 11 sets forth a deduced amino acid sequence of a portion of a HSP27 polypeptide corresponding with a sixth HSP27 epitope.

SEQ.ID NO: 12 sets forth a deduced amino acid sequence of a portion of a HSP27 polypeptide corresponding with a seventh HSP27 epitope.

SEQ.ID NO: 13 sets forth a deduced amino acid sequence of a portion of a HSP27 polypeptide corresponding with an eighth HSP27 epitope.

SEQ.ID NO: 14 sets forth a deduced amino acid sequence of a portion of a HSP27 polypeptide corresponding with a nineth HSP27 epitope.

SEQ.ID NO: 15 sets forth a polynucleotide sequence encoding a portion of a HSP27 polypeptide corresponding with the first HSP27 epitope.

SEQ.ID NO: 16 sets forth a polynucleotide sequence encoding a portion of a HSP27 polypeptide corresponding with the second HSP27 epitope.

SEQ.ID NO: 17 sets forth a polynucleotide sequence encoding a portion of a HSP27 polypeptide corresponding with the third HSP27 epitope.

SEQ.ID NO: 18 sets forth a polynucleotide sequence encoding a portion of a HSP27 polypeptide corresponding with the fourth HSP27 epitope.

SEQ.ID NO: 19 sets forth a polynucleotide sequence encoding a portion of a HSP27 polypeptide corresponding with the fifth HSP27 epitope.

SEQ.ID NO: 20 sets forth a polynucleotide sequence encoding a portion of a HSP27 polypeptide corresponding with the sixth HSP27 epitope.

SEQ.ID NO: 21 sets forth a polynucleotide sequence encoding a portion of a HSP27 polypeptide corresponding with the seventh HSP27 epitope.

SEQ.ID NO: 22 sets forth a polynucleotide sequence encoding a portion of a HSP27 polypeptide corresponding with the eighth HSP27 epitope.

SEQ.ID NO: 23 sets forth a polynucleotide sequence encoding a portion of a HSP27 polypeptide corresponding with the nineth HSP27 epitope.

SEQ.ID NO: 24 sets forth a deduced amino acid sequence of a mouse HSP25 polypeptide

SEQ.ID NO: 25 sets forth a deduced amino acid sequence of a portion of a HSP27 polypeptide.

SEQ.ID NO: 26 sets forth a deduced amino acid sequence of another portion of a HSP27 polypeptide.

SEQ.ID NO: 27 sets forth a deduced amino acid sequence of another portion of a HSP27 polypeptide.

SEQ.ID NO: 28 sets forth a deduced amino acid sequence of another portion of a HSP27 polypeptide.

SEQ.ID NO: 29 sets forth a deduced amino acid sequence of another portion of a HSP27 polypeptide.

SEQ.ID NO: 30 sets forth a deduced amino acid sequence of another portion of a HSP27 polypeptide.

SEQ.ID NO: 31 sets forth a deduced amino acid sequence of another portion of a HSP27 polypeptide.

SEQ.ID NO: 32 sets forth a deduced amino acid sequence of another portion of a HSP27 polypeptide.

SEQ.ID NO: 33 sets forth a deduced amino acid sequence of another portion of a HSP27 polypeptide.

SEQ.ID NO: 34 sets forth a polynucleotide sequence encoding a portion of a HSP27 polypeptide.

SEQ.ID NO: 35 sets forth a polynucleotide sequence encoding another portion of a HSP27 polypeptide.

SEQ.ID NO: 36 sets forth a polynucleotide sequence encoding another portion of a HSP27 polypeptide.

SEQ.ID NO: 37 sets forth a polynucleotide sequence encoding another portion of a HSP27 polypeptide.

SEQ.ID NO: 38 sets forth a polynucleotide sequence encoding another portion of a HSP27 polypeptide.

SEQ.ID NO: 39 sets forth a polynucleotide sequence encoding another portion of a HSP27 polypeptide.

SEQ.ID NO: 40 sets forth a polynucleotide sequence encoding another portion of a HSP27 polypeptide.

SEQ.ID NO: 41 sets forth a polynucleotide sequence encoding another portion of a HSP27 polypeptide.

SEQ.ID NO: 42 sets forth a polynucleotide sequence encoding another portion of a HSP27 polypeptide.

SEQ.ID NO: 43 sets forth a deduced amino acid sequence of another portion of a HSP27 polypeptide.

SEQ.ID NO: 44 sets forth a polynucleotide sequence encoding another portion of a HSP27 polypeptide.

Hereinafter are provided examples of specific implementations for performing the methods of the present disclosure, as well as implementations representing the compositions of the present disclosure. The examples are provided for illustrative purposes only, and are not intended to limit the scope of the present disclosure in any way.

### EXAMPLES

### Example 1—Preparation of a Substantially Pure HSP27 Polypeptide and HSP27 Polypeptide Fragment Preparation

Plasmids encoding HIS-tagged full-length HSP27 (HIS-tagged nucleic acid sequence: SEQ.ID NO: 3; nucleic acid sequence without HIS-tag: SEQ.ID NO: 1; HIS-tagged polypeptide sequence: SEQ.ID NO: 4; polypeptide sequence without HIS-tag: SEQ.ID NO: 2) and a HIS-tagged polypeptide fragment of HSP27 (SEQ.ID NO: 5), using a pET-21a vector, with the plasmids transformed into an Escherichia coli expression strain (DE3) as described by Raizman J. E. et al., 2013, Biochim Biophys Acta 2013, 1831 (12):1721-8. Recombinant proteins were purified with a Ni-NTA resin and Q-Sepharose™ (GE Healthcare). Endotoxin was removed by Pierce High-Capacity Endotoxin Removal Resin (ThermoFisher Scientific, Waltham, Mass., USA). The purity of the final rHSP27 and HSP27 rC1 protein preparations was determined to be more than 99% by SDS-PAGE with an endotoxin concentration lower than 2 units/mg protein measured by Limulus Amebocyte Lysate PYROGENT™ 125 Plus (Lonza).

It is noted that in this Example section and the Figures referenced therein, the produced HIS-tagged HSP27 polypeptide (SEQ.ID NO: 4) may be referred to as “rHSP27”. The produced HIS-tagged HSP27 fragment (SEQ.ID NO: 5) may be referred to as “rC1”)

### Example 2—Preparation of a Polyclonal Anti-HSP27 Antibody Preparation

A rabbit polyclonal antibody mimicking human HSP27 autoantibody was produced according to standard procedures by Cedarlane Laboratories, LTD (Burlington, ON) and in accordance with the requirements of the Canadian Council on Animal Care. Briefly, two rabbits were injected with 0.2 mg of rHSP27 (SEQ.ID NO: 2) (prepared as described in Example 1). After 28, 48 and 66 days the rabbits were boosted using 0.2 mg rHSP27 to increase the quantity of the resulting anti-HSP27 antibody. Rabbit sera was collected on day 78. The immunization efficiency was determined by an indirect ELISA coating the plates with rHSP27. The final serum was loaded to a 5 ml Protein G affinity column (GE Healthcare) for IgG affinity purification. After a 100 ml buffer A (50 mM PBS buffer containing 200 mM NaCl) wash, the antibody was eluted by buffer B (20 ml 20 mM sodium acetate, pH=2.5) and immediately neutralized by buffer C (400 mM PBS, pH=8.0). The antibody solution was then concentrated using a 15 ml of 30 kDa molecular weight cutoff filter (Millipore, Etobicoke, ON) and the buffer exchanged with buffer A for future usage. Two milligrams of biotinylated rHSP27 was then applied to a 1 ml streptavidin affinity column (GE Healthcare) for antigen affinity purification. The antigen conjugated column was washed with 20 ml Buffer A, loaded with PAb from the Protein G purification step and incubated for 5-10 min at 4° C. before washing twice with 20 ml buffer A. The antigen-specific anti-HSP27 antibody was then eluted with buffer B and immediately neutralized by buffer C to pH ˜7.0. The final purified polyclonal anti-anti-HSP27 antibody was buffer exchanged with DPBS buffer and filtered through 0.2 μm filter for future usage.

It is noted that in this Example section and the Figures referenced therein, the anti-HSP27 antibody may be referred to as “PAb”.

### Example 3—Preparation of Pharmaceutical Formulations Comprising HSP27 Polypeptides, and/or an Anti-HSP27 Antibody

The following are examples of pharmaceutical formulations comprising HSP27 polypeptide, anti-HSP27 antibodies and a mixture of HSP27 and anti-HSP27 antibodies:

TABLE 1
Example formulation HSP27 polypeptide
Pharmaceutical Formulation HSP27 polypeptide

| | | |
|---|---|---|
| HSP27 polypeptide | 100-250 | μg/ml |
| Mannitol (stabilizer) | 50-300 | mM |
| Histidine (buffer) | 10-50 | mM |

| | |
|---|---|
| Polysorbate 80 (surfactant) | 0.03-0.05% |
| Alum (adjuvant) | 0.2-0.8 mg Al³⁺/dose |
| pH | 6.2-6.8 |

TABLE 2
Example formulation Anti-HSP27 antibody
Pharmaceutical Formulation Anti-HSP27 antibody

| | | |
|---|---|---|
| Anti-HSP27 antibody | 25-100 | mg/ml |
| Mannitol (stabilizer) | 50-300 | mM |
| Histidine (buffer) | 10-50 | mM |

| | |
|---|---|
| Polysorbate 80 (surfactant) | 0.03-0.05% |
| pH | 6.2-6.8 |

TABLE 3
Example formulation HSP27/Anti-HSP27 antibody
Pharmaceutical Formulation HSP27/Anti-HSP27 antibody

| | | |
|---|---|---|
| Anti-HSP27 antibody | 25-100 | mg/ml |
| HSP27 polypeptide | 100-250 | μg/ml |
| Mannitol (stabilizer) | 50-300 | mM |
| Histidine (buffer) | 10-50 | mM |

| | |
|---|---|
| Polysorbate 80 (surfactant) | 0.03-0.05% |
| pH | 6.2-6.8 |

### Example 4—Functional Evaluation of HSP27 Polypeptides and Anti-HSP27 Antibodies (Membrane Binding)

The following example describes binding of HSP27 and an HSP27/anti-HSP27 Antibody Complex to Cellular Membranes.

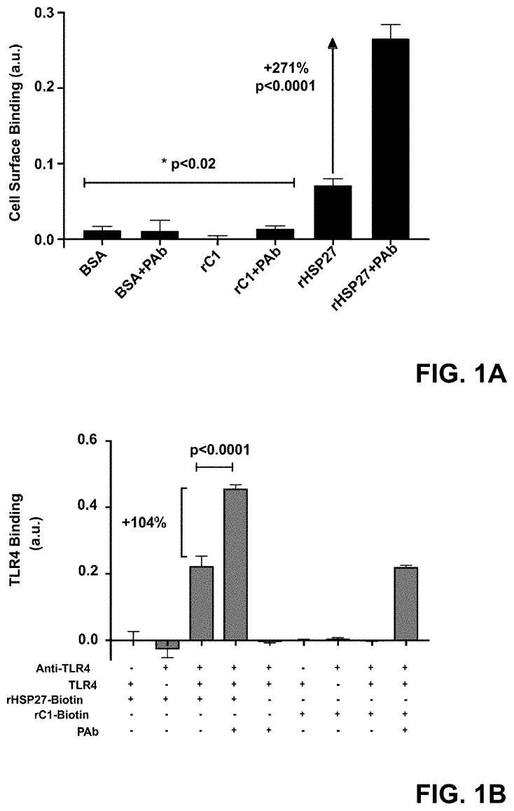

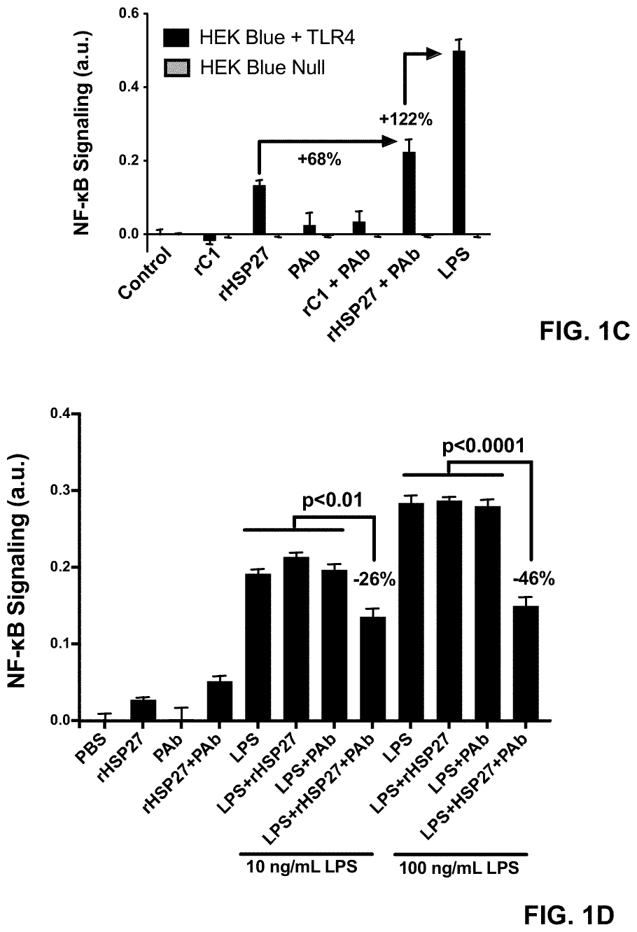

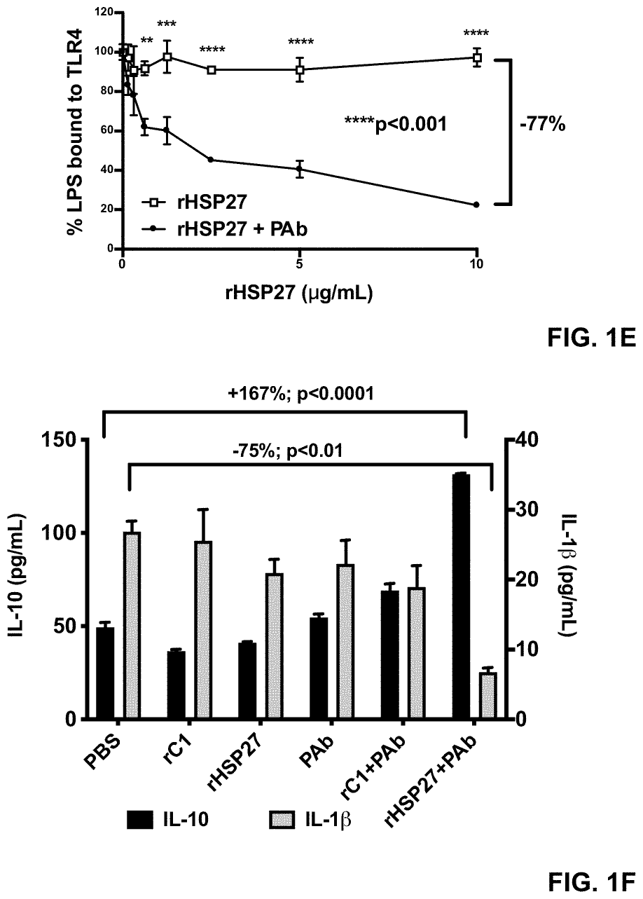

To determine the affinity of rHSP27 to bind to the cell membrane with or without PAb, THP-1 macrophage cells were incubated with the following biotin-labeled proteins plus streptavidin-HRP at 4° C. for 30 mins to prevent endocytosis of the reagents: BSA, rC1 and rHSP27 (SEQ.ID NO: 2). The cells were then washed thrice with cold PBS. The substrate TMB was then added to detect the retained HRP activity for 10 mins. The reaction was stopped by 2N H₂SO₄ and the color was quantified at 450 nm using a Synergy Mx plate reader (BioTek; Winooski, Vt.). The results are shown in FIG. 1A.

### Example 5—Functional Evaluation of HSP27 Polypeptides and Anti-HSP27 Antibodies (TLR4 Binding)

The following example describes binding of HSP27 and an HSP27/anti-HSP27 antibody complex to Toll Like Receptor TLR4.

An anti-TLR4 capture antibody (100 ng/well in carbonate-bicarbonate buffer; R&D Systems, Minneapolis, Minn.) with demonstrable specificity for TLR in vitro was coated onto NUNC maxisorp plates covered with an adhesive plastic and incubated overnight at 4° C. The coating solution was then removed, and the plate was blocked by adding 200 μl 1% BSA in PBST per well for at least 2 hrs at room temperature. Next, TLR4 was harvested from THP-1 macrophages as follows. Approximately 5×10⁷ THP-1 macrophage cells were re-suspended in 1 ml of 2% Trition-100/PBST, vigorously shaken for at least 2 hrs at 4° C., followed by the addition of 4 ml 1% BSA/PBS before being homogenized by sonication for 10 secs. After washing twice with 200 μl PBST, 100 ul of THP-1 macrophage lysate was added to the plate for an additional 1 hr incubation at room temperature to pull down TLR4. Finally, 100 μl of PBST containing 1% BSA with 1 μg/ml biotinylated rC1 or rHSP27 in the presence or absence of 5 μg/ml PAb and 1 μg/ml Strep-HRP was then applied for the interaction assay. After a 1 hr incubation at RT, the plate was washed 3 times with 200 μl/well, mixed with 100 μl of 3,3′,5,5′-tetramethylbenzidine (TMB; Millipore Sigma) and incubated for 10 mins at RT for development of the blue color. The reaction was stopped by the addition of 50 μl 2M H₂SO₄ and the optical density at 450 nm was read using a Synergy Mx plate reader. The results are shown in FIG. 1B.

### Example 6—Functional Evaluation of HSP27 Polypeptides and Anti-HSP27 Antibodies (NF-κ3 Signaling)

The following example describes NF-κB signaling initiated by HSP27 and an HSP27/anti-HSP27 antibody complex.

To test the impact of the HSP27 and an HSP27/anti-HSP27 antibody complex (referred to in the Example section and related figures as “[rHSP27+PAb] complex”) on inflammatory signaling two experiments were conducted, both involving cells containing an NF-κB reporter construct. These experiments were done with the previously reported knowledge that the amount of endotoxin contamination in these recombinant proteins is measurably low, and of no functional consequences—as the addition of polymyxin B did not alter the NF-κB signal (Raizman, J. E. et al., 2013, Biochimica et biophysica acta 1831, 1721-1728 (2013); published online EpubDec (10.1016/j.bbalip.2013.07.015); Li, C. et al., 2016 Scientific Reports 6, 36288; published online Epub11/08/online (10.1038/srep36288)). Lipopolysaccharide (LPS; from E. coli O111:64, Cedarlane;) the principal component of Gram-negative bacteria that activates the innate immune system via TLR4 was used as a comparative positive control for both experiments.

The first experiment (results, see: FIG. 1C) sought to confirm the specificity and requirement for signaling of TLR4 in NF-κB signaling; hence, Human embryonic kidney (HEK) Blue™ Null1-v (i.e., devoid of TLR4) and HEK Blue™ TLR4 (stably expressing TLR4, MD-2 and CD14 co-receptor genes in HEK Blue™ Null1-v parental line) cell lines (Invivogen) were used. Cells were subject to treatment for 24 hrs with rHSP27 (1 μg/mL), rC1 (1 μg/mL), PAb (5 μg/mL), rHSP27 (1 μg/mL) or rC1 (1 μg/mL) plus PAb (5 μg/mL) or LPS (10 ng/mL). The conditioned media from each treatment group was analyzed for the presence of secreted alkaline phosphatase (SEAP) using QUANTIBlue™ detection reagent (Invivogen) according to the manufacturer's instructions. Briefly, QUANTI-Blue™ detection reagent was mixed with cell supernatant (10:1) and incubated at 37° C. for up to 1 h. Optical absorbance at 620 nm was then measured using the BioTek Synergy Mx microplate reader. Media only controls (no cells) were assayed to ensure that there was no endogenous SEAP activity in the treatment media.

For the second experiment (results, see: FIG. 1D), NF-κB activation in THP1 XBlue™ cells (Invivogen) treated with PBS as a control were tested, and various combinations of rHSP27, PAb, and two concentrations of LPS (10 and 100 ng/ml), before assaying the conditioned media for SEAP.

### Example 7—Functional Evaluation of HSP27 Polypeptides and Anti-HSP27 Antibodies (Competition with LPS)

The following example describes competition of binding to TLR4 between HSP27 and an HSP27/anti-HSP27 antibody complex, and lipopolysaccharide (LPS).

To evaluate the competition between LPS and rHSP27 and [rHSP27+Pab] complex a competitive binding assay for TLR4 pulled down from the membrane fraction of THP-1 macrophages was conducted. Compared to rHSP27 alone, [rHSP27+PAb] reduced LPS binding to TLR4 by 77% (p<0.0001). The results are shown in FIG. 1E.

### Example 8—Functional Evaluation of HSP27 Polypeptides and Anti-HSP27 Antibodies (Cytokine Production)

The following example describes cytokine release upon binding of HSP27 and an HSP27/anti-HSP27 antibody complex to TLR4.

The downstream effects of the binding experiments of Examples 4-7 were assessed by evaluating THP-1 macrophage cytokine production. LPS-treated macrophage treated with rHSP27 and PAb released 167% higher levels of the anti-inflammatory cytokine IL-10 and 75% lower levels of the pro-inflammatory cytokine IL-1β compared to PBS control (p<0.001). The results are shown in FIG. 1F.

### Example 9—Functional Evaluation of HSP27 Polypeptides and Anti-HSP27 Antibodies (Binding with Scavenger Receptors CD36 and SR-AI)

The following example describes binding of HSP27 and an HSP27/anti-HSP27 antibody complex with scavenger receptors CD36 and SR-AI.

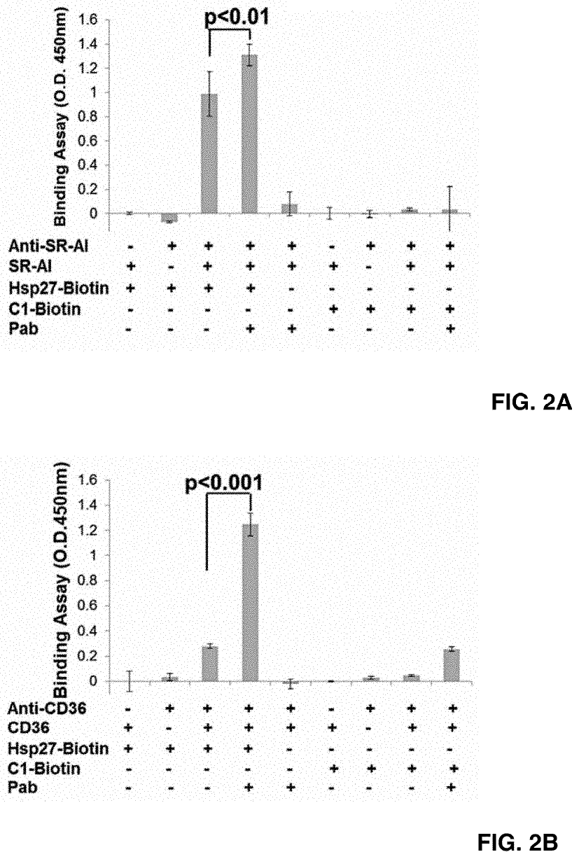

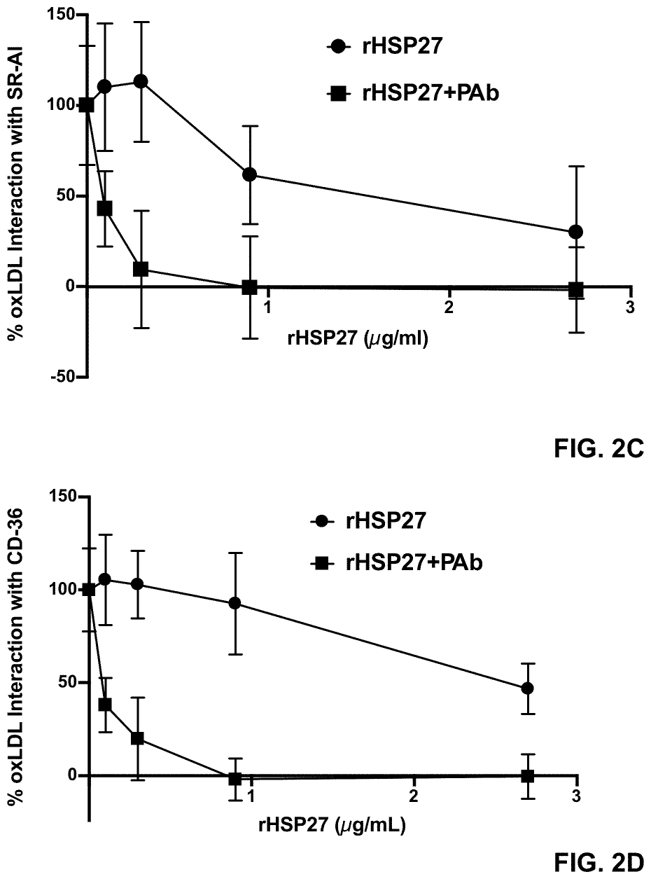

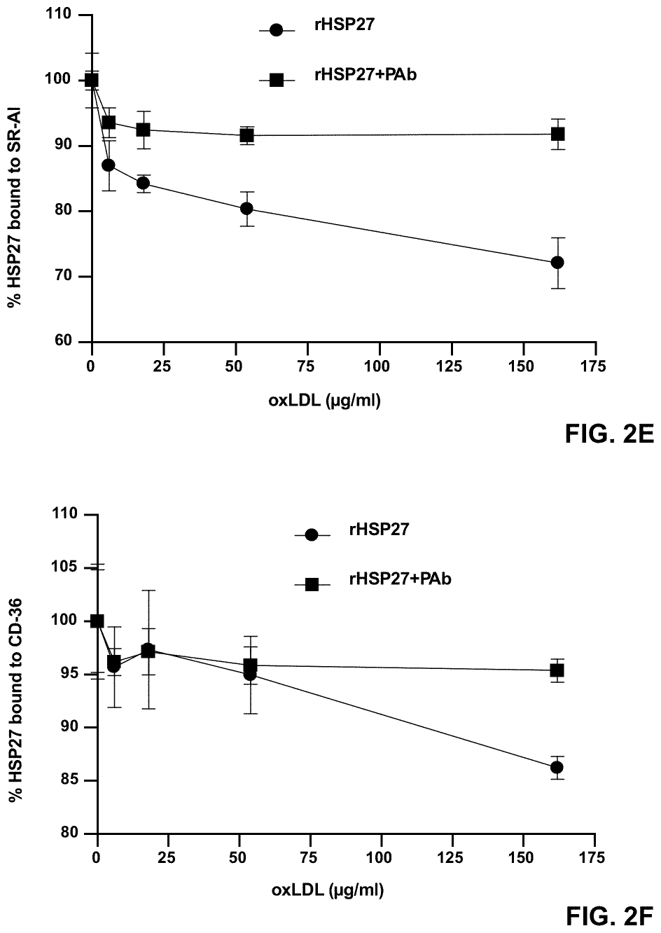

Macrophage membrane extracts or SR-AI and CD-36 were pulled down and their identities were verified by Western blotting. The PAb promoted the interaction between rHSP27 and the SR-AI and CD-36 receptors increasing the binding compared to rHSP27 without the PAb (p<0.01 and p<0.001 respectively). HSP27 fragment rC1 alone, PAb alone or samples without either the anti-SR-AI or anti-CD-36 antibodies, or the macrophage membrane protein fraction containing either SR-AI or CD-36 showed only background noise signals. Interestingly, the interaction between [rC1+Pab] and CD-36 was comparable to that of rHSP27 (alone)—perhaps indicating that it is the HSP27 alpha-crystallin domain (also found in rC1) that is important for the interaction with CD-36. Of note, as the ratio of rHSP27 to PAb is increased from 1:1 to 1:5, using excess PAb, the interaction with SR-AI and CD-36 increased by approximately 2.5- and 1.5-fold; respectively. The results are shown in FIGS. 2A-2B.

### Example 10—Functional Evaluation of HSP27 Polypeptides and Anti-HSP27 Antibodies (Binding Competition with oxLDL to Receptors CD36 and SR-AI)

The following example describes binding competition between HSP27 and an HSP27/anti-HSP27 antibody complex and oxLDL with scavenger receptors CD36 and SR-AI and binding competition.

As both scavenger receptors are involved in macrophage lipoprotein uptake it was assessed whether the [rHSP27+PAb] complex can compete for oxLDL binding to SR-AI or CD-36 pulled down from macrophage cell lysates. [rHSP27+PAb] complex was far superior to rHSP27 alone in competing with oxLDL for binding to SR-AI or CD36, with less than 0.5 μg/ml [rHSP27+PAb] complex almost completely nullifying oxLDL binding to either receptor. (see: FIGS. 2C-2D). The converse experiment was also carried out, using increasing concentrations of oxLDL to try to displace rHSP27 binding to either scavenger receptor. Essentially, [rHSP27+PAb] complex showed 100% binding to SR-AI or CD-36 regardless how high the concentration oxLDL was increased (to a maximum of 164 μg/ml; see: FIGS. 2E-2F)). In contrast, rHSP27 alone was easily displaced from SR-AI (e.g., a 50% reduction in rHSP27 binding at a low concentration of oxLDL), but less so from CD-36 except at higher oxLDL concentrations (e.g., 40% displacement at 164 μg/ml oxLDL). Together, these results show that PAb potentiated the interaction of rHSP27 with both SR-AI and CD36, which attenuated oxLDL binding to its cognate receptors.

### Example 11—Functional Evaluation of HSP27 Polypeptides and Anti-HSP27 Antibodies (Attenuation of oxLDL Uptake by Receptors CD36 and SR-AI)

The following example describes the attenuation of oxLDL uptake in the presence of HSP27 and an HSP27/anti-HSP27 antibody complex by scavenger receptors CD36 and SR-AI and binding competition.

To assess the relative contribution of SR-AI and CD36 in oxLDL uptake a HEK Blue™ Null1-v cell line which is devoid of both of these receptors was used, and then separately transfected with SR-AI and CD-36 into these parental cells. After confirming the expression of these scavenger receptors and comparing their abundance relative to THP-1 macrophage using both Western blotting and FACS (the HEK Blue™ cells expressing SR-AI or CD-36 were then treated with Dil-oxLDL and combinations of rHSP27, rC1 and PAb for 2 hrs before performing FACS. For each experimental condition, the percentage of fluorescent cells was measured, as well as the MFI, a reflection of the amount of oxLDL that was taken up per experimental condition. HEK Blue™ Null-v cells served as negative controls and showed low (background) levels of fluorescence for PBS and all other treatments, thereby highlighting the requirement for SR-AI and CD-36 for oxLDL uptake.

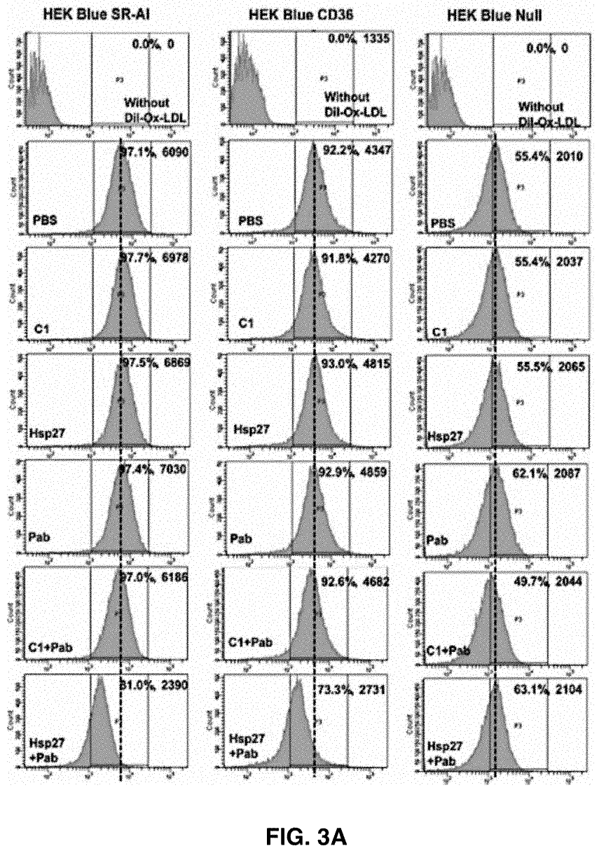

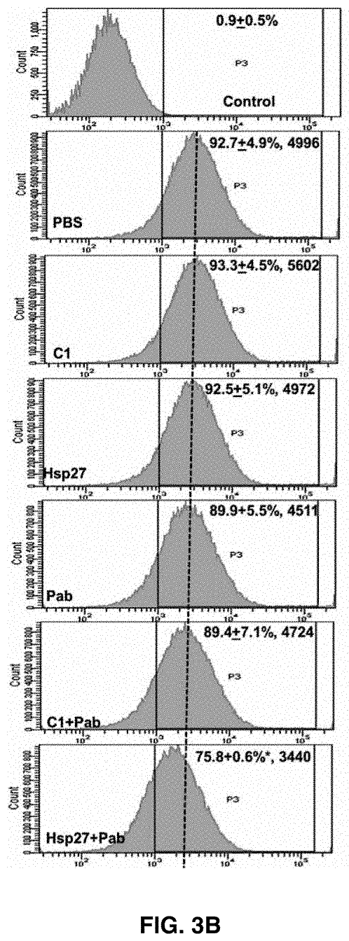

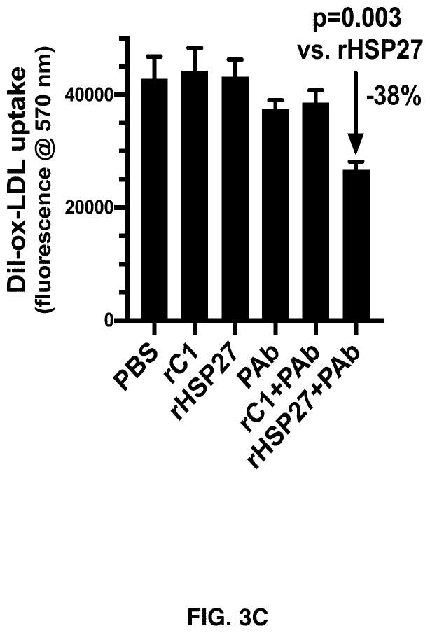

For HEK Blue™ SR-AI cells the percentage of positive cells was very similar for all control conditions (PBS: 97.1%, rC1: 97.7%, Pab: 97.4%), as well as treatment with rHSP27 alone (97.5%). In contrast, the [rHSP27+PAb] complex had 81.0% positive cells, reflecting a modest drop in the percentage of cells that took up oxLDL. While the MFI was similar amongst the control treatments, the [rHSP27+PAb] complex reduced oxLDL uptake by 65.2% (6,869 vs. 2,390 a.u.) in the HEK Blue™ cells expressing SR-AI. Compared to PBS control, the presence of [rHSP27+PAb] complex reduced Dil-oxLDL uptake by 61% and 37% from SR-AI and CD36 expressing cells, respectively, whereas the other control treatments had negligible effects (see: FIG. 3A).

For the HEK Blue™ CD-36 cells, there was no significant difference in percentage of cells with oxLDL uptake for the control treatments (e.g., PBS 92.2%, rC1 91.8%, PAb 92.9%). However, the percentage of cells with oxLDL uptake for the [rHSP27+PAb] complex treatment group was only 73.3% compared to the cells treated with rHSP27 alone (93.0%). With regard to the MFI signal, the cells treated with [rHSP27+PAb] complex was 43.3% lower than those treated with rHSP27 alone (4,815 vs. 2,731 a.u., FIG. 3A).

The question that was then addressed was whether in THP-1 macrophages, the competitive binding of the [rHSP27+PAb] complex affects oxLDL uptake using both FACS and a plate reader assay. Dil-oxLDL uptake was reduced in the presence of [rHSP27+PAb] complex by both techniques:
a) For the FACS studies, the percentage of cells that were fluorescent for Dil-oxLDL was similar amongst the controls: PBS (92.7%), rC1 (93.3%) and PAb (89.95), but dropped to 75.8% with [rHSP27+PAb] complex treatment (see: FIG. 3B). With regards to the MFI signal that reflects the amount of Dil-oxLDL uptake for each experimental condition, the rHSP27 (alone) treatment group (4,972) was indistinguishable from the PBS control (4,996 a.u.). However, macrophage cells treated with [rHSP27+PAb] complex had a 31% lower MFI signal (3,440 a.u) compared to rHSP27 alone.
b) A plate reader assay showed a similar 38% reduction of Dil-oxLDL uptake with the [rHSP27+PAb] complex, compared to rHSP27 alone (p=0.003), and no significant changes with rC1, rHSP27, PAb alone, or rC1 and PAb (see: FIG. 3C).

### Example 12—Functional Evaluation of HSP27 Polypeptides and Anti-HSP27 Antibodies (Limitation of Pathogenesis of Non-Alcoholic Fatty Liver Disease)

The following example describes how pharmaceutical formulations comprising HSP27 polypeptides, and anti-HSP27 antibodies can limit the pathogenesis of NASH.

Several observations support the central role of oxidized low-density lipoprotein (oxLDL) in hepatic inflammatory and fibrotic responses. The major detrimental effects of oxLDL are mediated by Kupffer cells (KCs), the macrophages of the liver. Indeed, KCs are clearly associated with the pathogenesis of NASH (Baffy, G., 2009, J Hepatol. 2009, 51: 212-223). In the livers of hyperlipidemic mice, for example, the formation of bloated foamy KCs is associated with hepatic inflammation, and there is increasing evidence for the role of oxLDL in this process (Wouters K. et al., 2008, Hepatology 48: 474-486). Moreover, targeting oxLDL with specific immunization strategies against oxLDL reduces NASH in mice (Bieghs V. et al., 2012, Hepatology, 56: 894-903). Therefore, oxLDL-induced activation of KCs can be considered as a mechanism by which inflammation and fibrosis arise in NASH.

Internalization of modified lipids in KCs is thought to be mediated by the scavenger receptors (SRs): i) scavenger receptor A (SR-AI) and ii) cluster of differentiation 36 (CD36) (Kunjathoor V. V. et al., 2002, J Biol. Chem. 277: 49982-49988). Both receptors are involved in oxLDL internalization, and protein levels of both receptors are increased after oxLDL incubation in macrophages (Yoshida H. et al., 1998, Arteriosclerosis, thrombosis, and vascular biology, 18: 794-802). Furthermore, hyperlipidemic mice containing a hematopoietic deletion of both SR-A and/or CD36 have reduced levels of hepatic inflammation and fibrosis, indicating that SR-mediated internalization of modified lipids by KCs is an important trigger to develop NASH (Bieghs V. et al., 2010, Gastroenterology, 138: 2477-2486, 2486.e2471-2473).

Thus, pharmaceutical formulations comprising at least one of HSP27 or anti-HSP27 antibodies when delivered to liver cells, notably Kupffer cells, can bind to the SR-A and CD36 receptors and attenuate the binding of oxLDL to the same receptors (as shown in Examples 9-11), and limit internalization of oxLDL in the Kupffer cells. This in turn can restrict an inflammatory reaction (as shown in Example 8) and fibrosis, and the pathogenesis of NASH.

### Example 13—Characterization of Anti-HSP27 Antibodies

The following example describes the characterization on anti-HSP27 antibodies via epitope mapping.

To characterize the polyclonal anti-HSP27 antibodies (PAb) (see: Example 2) we performed epitope mapping on the HSP27 protein using synthesized HSP27 peptides affixed to a membrane (i.e., the spot blot peptide technique) and compared these epitope mapping results to those obtained when human serum was added (Li. C. et al., Scientific Reports, 2016, 6, 36288). A spot blot membrane was constructed with 49 spots representing overlapping 15 amino acid peptide sequences of the full-length HSP27 protein. These peptides were synthesized on a modified cellulose membrane (Kinexus Bioinformatics Corporation, Vancouver, BC). The membrane was soaked ON in 20 mL of PBS containing 1% BSA (pH 7.5), and then washed three times with PBST. A 1:200 (v/v) diluted plasma was added, and the membrane was incubated for 2 hrs at RT. After three washes with PBST, the membrane was incubated in a 1:4,000 dilution of mouse monoclonal anti-human IgG-HRP (Jackson Immunoresearch) for 1 hr. After three additional washes with PBST, the interacting peptide dots were developed with an ECL kit (GE Healthcare) for 3-5 mins according to manufacturer's instructions and imaged by the FluorChem® Q Imaging System (Cell Biosciences, Santa Clara, Calif.).

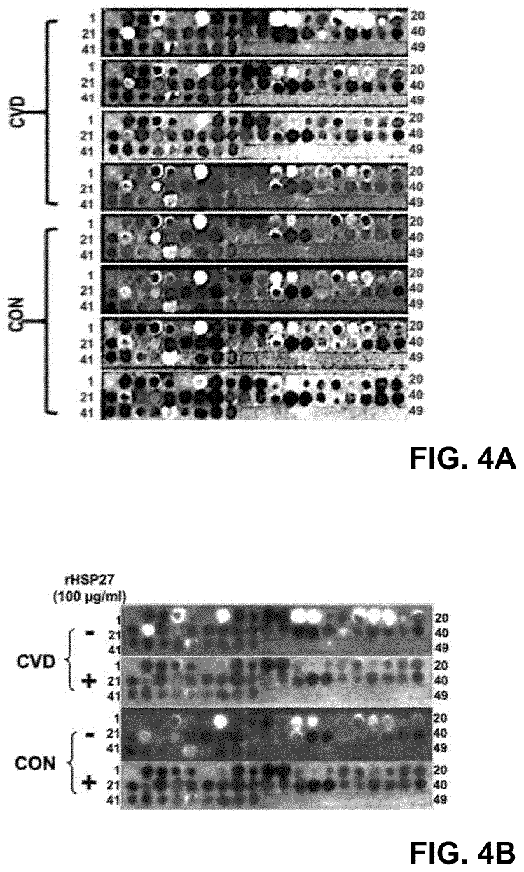

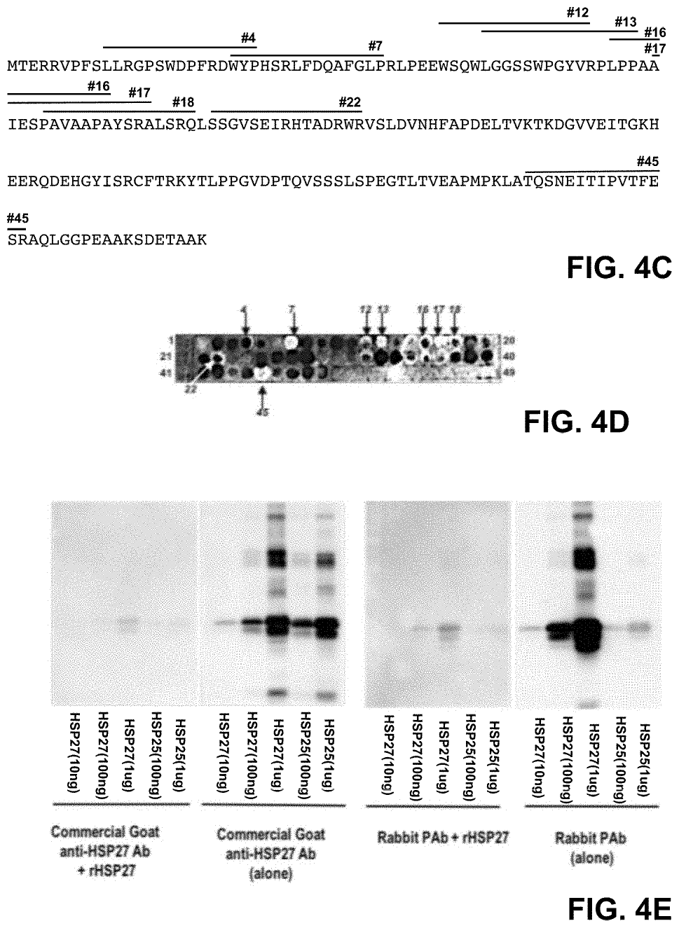

Specific 15-mers of the linearized protein reacted with the PAb in a pattern that was identical to that of the naturally occurring AAbs—whether they were derived from the blood of 4 healthy subjects (CON) or 4 cardiovascular disease patients (CVD) (FIG. 4A). The binding of the PAb to these epitopes was abrogated by the addition of rHSP27 (100 μg/ml vs. an irrelevant anti-human IgG negative control), thereby indicating that epitopes in the full-length rHSP27 were similar to those on the spot blot (FIG. 4B). A summary of the HSP27 epitopes is schematically outlined in FIG. 4C. When the rabbit PAb was applied to the spot blot the identified epitopes were identical to those note with human sera from CON and CVD subjects, thereby confirming the specificity of the PAb (FIG. 4D). Finally, the immunoreactivity of the rabbit PAb was compared vs. a commercial (goat) anti-HSP27 antibody using Western blotting of rHSP27 and rHSP25 (FIG. 4E). The addition of excess rHSP27 essentially eliminated an interaction signal for either antibody. In the absence of exogenous rHSP27, the band recognition pattern for various (loaded) amounts of rHSP27 (10 ng, 100 ng, 1 μg) or rHSP25 (100 ng, 1 μg) were very similar for these two antibodies.

### Example 14—Attenuation of Hepatic Inflammation and Fibrosis in Mice

The following example describes the attenuation of hepatic inflammation and fibrosis in mice having been administered the mouse ortholog of HSP27, known as HSP25 (SEQ.ID NO: 24).

Female APO*3Leiden.CETP mice (on a C57BL/6 background) were bred at the animal facility of the Netherlands Organization (TNO) for Applied Scientific Research and fed a Western-type diet prior to being assessed for study inclusion. Female mice were used as they more readily develop elevated plasma levels of cholesterol and triglycerides, as well as atherosclerosis, when compared to males fed a cholesterol-containing diet. Approximately 20-25% of these mice do not respond to cholesterol-enriched diets. Based on plasma cholesterol and triglyceride levels mice that responded to a cholesterol enriched diet by showing elevated lipoprotein levels were selected for this study and transferred to the University of Calgary where the institutional Animal Care Committee had already approved the study protocol (MC17-0015). The mice were initially housed in quarantine and immediately started on a high fat diet (HFD, 15.8% fat, 1.25% cholesterol, Envigo-Teklad diet #TD94059) for a 6 week run-in period. Four mice were then euthanized and underwent histological assessment of their liver and aortae for evidence of fat infiltration and atherogenesis, respectively. An additional 32 mice were randomly assigned to two groups that received 11 weekly subcutaneous vaccinations with either rHSP25 (SEQ.ID NO: 24) (n=16) or rHSP27 fragment rC1 (SEQ.ID NO: 5) (n=16) before being euthanized one week after the last injection. It is noted that rHSP27 lacks all but one of the epitopes identified in Example 13, and the remaining epitope rHSP27 epitope varies from the corresponding rHSP25 sequence.

Vaccination was performed by mixing rHSP25 (100 μg or ˜4 nmol in a volume of 75 μL) or rHSP27 fragment rC1 (100 μg or ˜7 nmol in a volume of 75 μL) with an aluminum hydroxide gel adjuvant (Alhydrogel® 2%, Al, 25 μL; Invivogen vac-alu-250, San Diego, Calif.). Hence, the molarity of the rHSP27 fragment rC1 vaccine was almost twice that of the rHSP25 vaccine.

Weekly blood samples were collected from the saphenous vein of these mice and stored at −80° C. for future use. At the end of the study blood samples were obtained via the right heart ventricle after 16 hrs of overnight fasting. This was followed by whole body perfusion with PBS. The left liver lobe was then flash frozen in liquid nitrogen and stored at −80° C. for gene expression assays. Thereafter, mice were systemically perfused with 10% NBF via the heart left ventricle, and the heart, aorta and liver tissues were removed and immersed in 10% NBF overnight.

Various histopathological parameters were subsequently evaluated, notably fatty lesion area size, liver macrophage content, hepatic fibrosis, and expression of inflammatory cytokines, using the following methodologies and techniques.

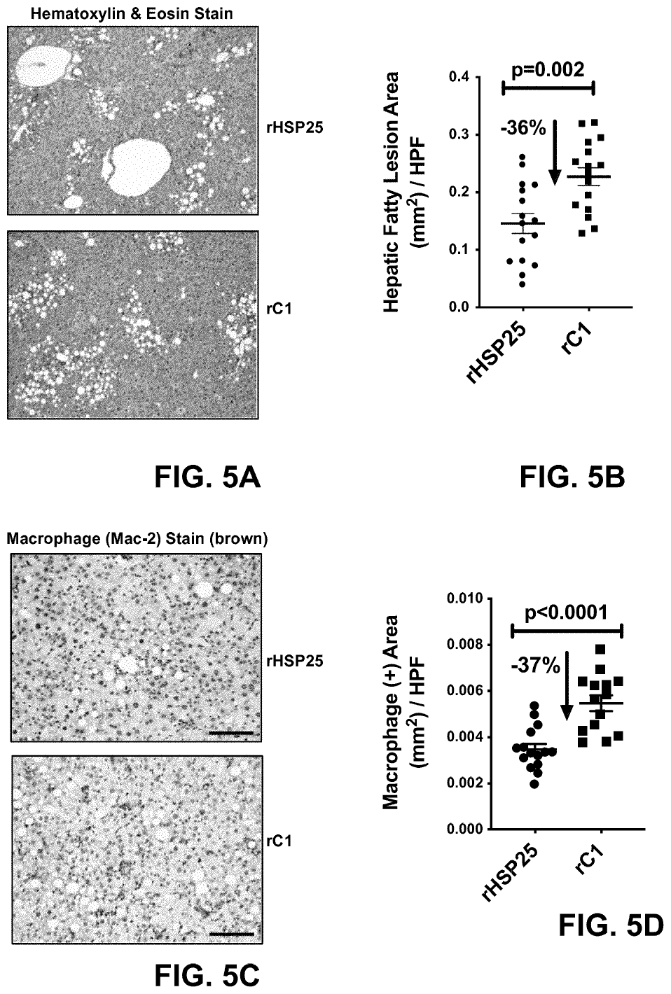

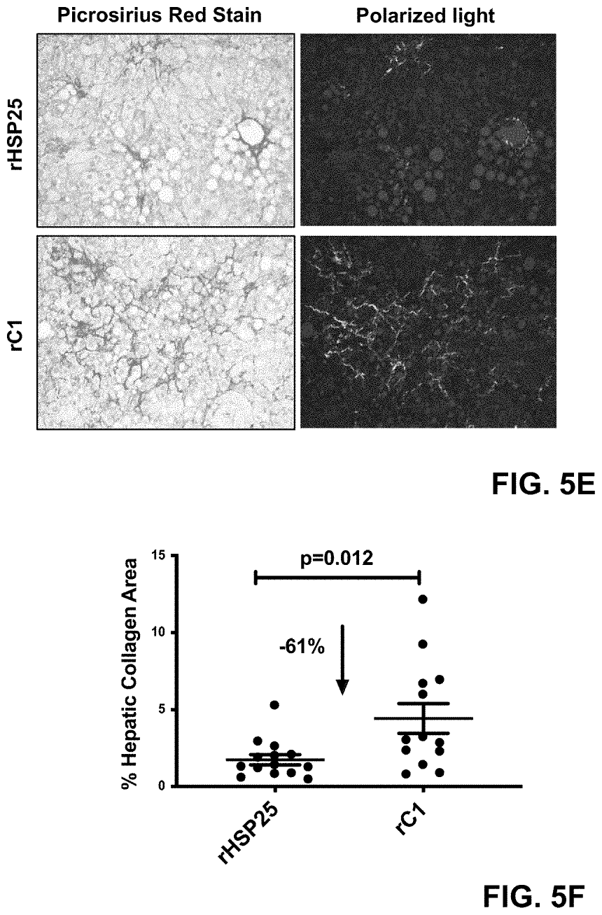

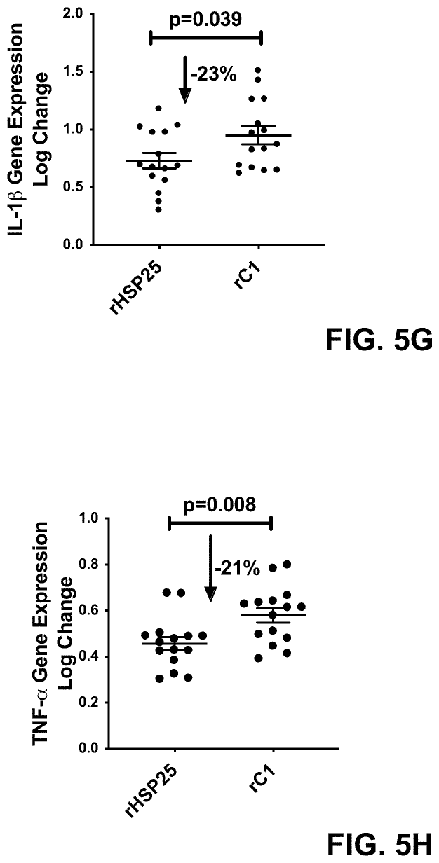

After fixation in 10% NBF, liver tissue was embedded in paraffin, and serially sectioned at 5-μm intervals. After deparaffinization, tissue sections were rehydrated and subjected to H&E staining. Histopathological changes of liver tissue based on H&E staining were examined and photographed with an Olympus BX52 microscope (FIG. 5A). Immunolabelling for macrophages was performed using a rat anti-Mac-2 antibody (1:500; CedarLane Laboratories, Burlington, ON; CL8942AP) (FIG. 5C). Sections of liver tissue were deparaffinized in xylene and rehydrated in graded concentrations of ethanol/water. The sections were blocked with 10% normal horse serum followed by incubation with primary antibody at 4° C. overnight. A peroxidase conjugated anti-rat secondary antibody was then applied (1:100, Vector Laboratories). Endogenous peroxidase activity was quenched with 3% H₂O₂. Antibody reactivity was detected with an ABC kit (Vector Laboratories) and visualized with diaminobenzidine (DAB). Sections were counterstained with hematoxylin, cleared, and mounted on glass slides. Immunopositive areas in cross sections were quantified using Image-Pro software according to previously described methods (Seibert, T. A. et al., 2013, 62:1446-1454). The negative control consisted of substituting IgG for the primary antibody. Picrosirius red was used to stain for collagen as previously described (Puchtler H. et al., 1973, FBA. Beitr Pathol 1973; 150:174-87; and Junqueira L. C. et al., 1979, Histochem J. 1979; 11:447-55). Collagen bundles were defined as bright yellow, if observed using light microscopy, or, if observed under polarizing microscope, orange (FIG. 5E). The collagen areas and lesion areas in cross sections were quantified using Image-Pro software according to previously described techniques Hibbert B. et al., 2004, Am J Physiol Heart Circ Physiol, 287:H518-H524.

The expression of the inflammatory MΦ markers IL1β and TNF1α was assessed in murine liver tissue from 15 mice per group. Total RNA was isolated from a piece of the liver (100 mg) using TRIzol reagent (Thermo Fisher Scientific) and 1 μg of purified RNA was reverse transcribed to cDNA using the qScript cDNA SuperMix (Quantabio, 95048-100) mastermix. Quantitative real-time PCR was performed using PerfeCta SYBR Green Supermix ROX (Quantabio, 95055-500) on the StepOnePlus Real-Time PCR System (Thermo Fisher Scientific). β-Actin was used as an endogenous control to calculate fold changes of the target gene expression using the 2^{-ΔΔCt} method.

Statistical methods involved the expression of continuous variables as mean±standard deviation and the comparison of unpaired treatment groups which was performed using a Student's t-test. Quantitative PCR gene expression data were log-transformed and are reported as log-fold change in order to more normally distribute the data and permit parametric statistical analyses. All analyses were performed in a blinded fashion using GraphPad Prism 8 (GraphPad Software, La Jolla, Calif.). A two-tailed a level of 0.05 was used to define statistical significance.

The following quantitative results were obtained.

The fatty lesion area was reduced by 36% in the livers of the rHSP25 vs. rHSP27 fragment rC1 vaccinated mice (0.146±0.070 vs. 0.227±0.062; respectively, p=0.002) (see: FIG. 5B). Similarly, the hepatic area occupied by macrophages was lowered by 37% in the rHSP25 vs. rHSP27 fragment rC1 vaccinated mice (0.003±0.001 vs. 0.005±0.001; respectively, p<0.0001) (see: FIG. 5D). With regards to hepatic fibrosis, there was a 61% reduction in collagen accumulation (rHSP25: 1.74±1.25 vs. rC1: 4.43±3.49; p=0.012) (See: FIG. 5F).

Finally, compared to rHSP27 fragment rC1, vaccination with rHSP25 produced marked reductions in the hepatic expression of inflammatory cytokines (as determined using qPCR, and log transformed): i) IL-1β (0.730±0.260 vs. 0.950±0.300; respectively, p=0.039) (see: FIG. 5G) ii) TNFα (0.457±0.111 vs. 0.580±0.123; respectively, p=0.008) (see: FIG. 5H)

Taken together, the foregoing results are consistent with certain aspects of the present disclosure, namely that pharmaceutical formulations comprising an HSP27 polypeptide can limit the pathogenesis of NASH.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1-28. (canceled)

29. A method for treating non-alcoholic fatty liver disease, the method comprising administering to a human subject in need thereof a pharmaceutical formulation comprising at least one of (i) an HSP27 polypeptide or an immunologically equivalent portion thereof, or (ii) an anti-HSP27 antibody or functional antibody fragment thereof, wherein the pharmaceutical formulation is administered in an effective amount to treat non-alcoholic fatty liver disease in the subject.

30. A method according to claim 29, wherein the HSP27 polypeptide or the immunologically equivalent portion thereof is a polypeptide encoded by a nucleic acid sequence selected from the nucleic acid sequences consisting of:
(a) SEQ.ID NO: 1; SEQ.ID NO: 15; SEQ.ID NO: 16; SEQ.ID NO: 17; SEQ.ID NO: 18; SEQ.ID NO: 19; SEQ.ID NO: 20; SEQ.ID NO: 21; SEQ.ID NO: 22; and SEQ.ID NO: 23;
(b) a nucleic acid sequence that is substantially identical to SEQ.ID NO: 1; SEQ.ID NO: 15; SEQ.ID NO: 16; SEQ.ID NO: 17; SEQ.ID NO: 18; SEQ.ID NO: 19; SEQ.ID NO: 20; SEQ.ID NO: 21; SEQ.ID NO: 22; and SEQ.ID NO: 23;
(c) a nucleic acid sequence that is substantially identical to SEQ.ID NO: 1; SEQ.ID NO: 15; SEQ.ID NO: 16; SEQ.ID NO: 17; SEQ.ID NO: 18; SEQ.ID NO: 19; SEQ.ID NO: 20; SEQ.ID NO: 21; SEQ.ID NO: 22; and SEQ.ID NO: 23 but for the degeneration of the genetic code;
(d) a nucleic acid sequence that is complementary to SEQ.ID NO: 1; SEQ.ID NO: 15; SEQ.ID NO: 16; SEQ.ID NO: 17; SEQ.ID NO: 18; SEQ.ID NO: 19; SEQ.ID NO: 20; SEQ.ID NO: 21; SEQ.ID NO: 22; and SEQ.ID NO: 23;
(e) a nucleic acid sequence encoding a polypeptide having the amino acid sequence set forth in SEQ.ID NO: 2, SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14;
(f) a nucleic acid sequence that encodes an immunologically equivalent functional variant of the amino acid sequence set forth in SEQ.ID NO: 2, SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14; and
(g) a nucleic acid sequence that hybridizes under stringent conditions to any one of the nucleic acid sequences set forth in (a), (b), (c), (d), (e), or (f).

31. A method according to claim 29, wherein the immunologically equivalent portion of the HSP27 polypeptide comprises at least 30, at least 40, at least 50, at least 60, at least 70, at least 80, at least 90, at least 100, or 105 consecutive amino acids of the 105 N-terminal amino acid residue portion of SEQ.ID NO: 2.

32. A method according to claim 29, wherein the immunologically equivalent portion of the HSP27 polypeptide is a polypeptide comprising at least two, at least three, at least four, at least five, at least six, at least seven at least eight, or all nine of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14.

33. A method according to claim 29, wherein the immunologically equivalent portion of the HSP27 polypeptide is a polypeptide comprising (a) (i) at least two of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, and (ii) at least 30 consecutive amino acids of the 105 N-terminal amino acid residue portion of SEQ.ID NO: 2, or (b) (iii) at least three of SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and (iv) SEQ.ID NO: 14, and at least 50 consecutive amino acids of the 105 N-terminal amino acid residue portion of SEQ.ID NO: 2.

34. A method according to claim 29, wherein the immunologically equivalent portion of the HSP27 polypeptide comprises a peptide selected from SEQ.ID NO; 25; SEQ.ID NO: 26; SEQ.ID NO; 27; SEQ.ID NO: 28; SEQ.ID NO; 29; SEQ.ID NO: 30; SEQ.ID NO; 31; SEQ.ID NO: 32; and SEQ.ID NO: 33.

35. A method according to claim 29, wherein the anti-HSP27 antibody is a polyclonal anti-HSP27 antibody, optionally a monoclonal anti-HSP27 antibody, optionally belonging to the IgG class of antibodies.

36. A method according to claim 29, wherein the anti-HSP27 antibody is an antibody binding to at least one HSP27 epitope selected from: SEQ.ID NO: 6; SEQ.ID NO: 7; SEQ.ID NO: 8; SEQ.ID NO: 9; SEQ.ID NO: 10; SEQ.ID NO: 11; SEQ. ID NO: 12; SEQ.ID NO: 13; and SEQ.ID NO: 14.

37. A method according to claim 29, wherein the pharmaceutical formulation comprises an HSP27 polypeptide or an immunologically equivalent portion thereof and an adjuvant, wherein the pharmaceutical formulation does not include the anti-HSP27 antibody or a functional antibody fragment thereof.

38. A method according to any one of claim 29, wherein the pharmaceutical formulation comprises an HSP27 polypeptide or an immunologically equivalent portion thereof and an adjuvant, and not the anti-HSP27 antibody or a functional antibody fragment thereof, the HSP27 polypeptide or immunologically equivalent portion thereof eliciting an immune response in the subject and the production of native anti-HSP27 antibodies in the blood serum of the subject, the HSP27 polypeptide or immunologically equivalent portion thereof and native anti-HSP27 antibodies forming polypeptide/antibody complexes, wherein said complexes bind to the cell membrane of macrophage cells or liver cells in the subject to thereby trigger an anti-inflammatory response.

39. A method according to claim 29, wherein the pharmaceutical formulation comprises an anti-HSP27 antibody, or a functional fragment thereof, wherein the formulation does not include the HSP27 polypeptide or an or immunologically equivalent portion thereof.

40. A method according to claim 29, wherein the pharmaceutical formulation comprises an anti-HSP27 antibody, or a functional fragment thereof, and does not include the HSP27 polypeptide or immunologically equivalent portion thereof, wherein upon administration a polypeptide/antibody complex is formed between the anti-HSP27 antibody or a functional fragment thereof and the native HSP27 polypeptide of the subject in the blood serum of the subject, and wherein said complex binds to the cell membrane of macrophage cells or liver cells in the subject to thereby trigger an anti-inflammatory response.

41. A method according to claim 29, wherein the pharmaceutical formulation comprises an anti-HSP27 antibody, or a functional fragment thereof, and does not include the HSP27 polypeptide or immunologically equivalent portion thereof, wherein upon administration a complex is formed between the anti-HSP27 antibody or a functional fragment thereof, and the native HSP27 of the subject in the blood serum of the subject, wherein the formed complex limits the interaction of circulating oxidized low density lipoprotein (oxLDL) with at least one of liver cell scavenger receptors SR-A1 or CD-36, to thereby restrict uptake of the circulating oxLDL by the liver cells of the subject.

42. A method according to claim 29, wherein the pharmaceutical formulation comprises an HSP27 polypeptide or immunologically equivalent portion thereof and an anti-HSP27 antibody or anti-HSP27 functional antibody fragment thereof, the HSP27 polypeptide or immunologically equivalent portion thereof and the native anti-HSP27 antibodies forming polypeptide/antibody complexes or polypeptide/anti-HSP27 functional antibody fragment complexes, the complexes being present in the formulation, or being formed in vivo in the blood serum of the subject upon administration, wherein said complexes bind to the cell membrane of macrophage cells or liver cells in the subject to thereby trigger an anti-inflammatory response.

43. A method according to claim 29, wherein the pharmaceutical formulation comprises an HSP27 polypeptide or immunologically equivalent portion thereof and an anti-HSP27 antibody or anti-HSP27 functional antibody fragment thereof, the HSP27 and native anti-HSP27 antibodies forming polypeptide/antibody complexes or polypeptide/anti-HSP27 functional antibody fragment complexes, the complexes being present in the formulation, or being formed in vivo in the in the blood serum of the subject upon administration, wherein the interaction of circulating oxLDL with at least one of liver cell scavenger receptors SR-A1 or CD-36 is reduced, to thereby restrict uptake of the circulating oxLDL by the liver cells of the subject.

44. A method according to claim 29, wherein the pharmaceutical formulation comprises the HSP27 polypeptide or immunologically equivalent portion thereof in a concentration ranging from about 100 μg/ml to about 10 mg/ml, or the anti-HSP27 antibody or functional antibody fragment thereof in a concentration ranging from about 25 mg/ml to about 200 mg/ml.

45. A method according to claim 29, wherein the pharmaceutical formulation comprises a HSP27 polypeptide or an immunologically equivalent portion thereof, and a dose ranging from about 100 μg to about 1 mg is administered to the human subject, or the pharmaceutical formulation comprises an anti-HSP27 antibody or a functional antibody fragment thereof, and a dose ranging from about 100 mg to about 5 g is administered to the human subject.

46. A method according to claim 29, wherein the pharmaceutical formulation further comprises a pharmaceutically acceptable carrier, excipient or diluent.

47. A method according to claim 29, wherein the pharmaceutical formulation is administered parenterally.

48. A method according to claim 29, wherein the non-alcoholic fatty liver disease is non-alcoholic steatohepatitis, optionally characterized by hepatic fibrosis or hepatic cirrhosis.


SEQUENCE LISTING (44 sequences)

SEQ ID NO: 1 - DNA, 624 bp, Homo sapiens
atgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaagaattc

SEQ ID NO: 2 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 3 - DNA, 675 bp, Homo sapiens
catatgcatcaccatcatcaccaccaccatgatgacgacgacaagaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaagaattcagcagcggg

SEQ ID NO: 4 - protein, 218 aa, Homo sapiens
MHHHHHHHHDDDDKTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 5 - protein, 129 aa, Homo sapiens
MHHHHHHHHDDDDKGGDRWRVSLDVNHFAPDELTVKTKDGVVEITGKHEERQDEHGYISRCFTRKYTLPPGVDPTQVSSSLSPEGTLTVEAPMPKLSTQSNEITIPVTFESRAQLGGPEAAKSDETAAK

SEQ ID NO: 6 - protein, 15 aa, Homo sapiens
LLRGPSWDPFRDWYP

SEQ ID NO: 7 - protein, 15 aa, Homo sapiens
WYPHSRLFDQAFGLP

SEQ ID NO: 8 - protein, 15 aa, Homo sapiens
WSQWLGGSSWPGYVR

SEQ ID NO: 9 - protein, 15 aa, Homo sapiens
LGGSSWPGYVRPLPP

SEQ ID NO: 10 - protein, 15 aa, Homo sapiens
LPPAAIESPAVAAPA

SEQ ID NO: 11 - protein, 15 aa, Homo sapiens
AIESPAVAAPAYSRA

SEQ ID NO: 12 - protein, 15 aa, Homo sapiens
PAVAAPAYSRALSRQ

SEQ ID NO: 13 - protein, 15 aa, Homo sapiens
SSGVSEIRHTADRWR

SEQ ID NO: 14 - protein, 15 aa, Homo sapiens
TQSNEITIPVTFESR

SEQ ID NO: 15 - DNA, 45 bp, Homo sapiens
ctcctgcggggccccagctgggaccccttccgcgactggtacccg

SEQ ID NO: 16 - DNA, 45 bp, Homo sapiens
tggtacccgcatagccgcctcttcgaccaggccttcgggctgccc

SEQ ID NO: 17 - DNA, 45 bp, Homo sapiens
tggtcgcagtggttaggcggcagcagctggccaggctacgtgcgc

SEQ ID NO: 18 - DNA, 45 bp, Homo sapiens
ttaggcggcagcagctggccaggctacgtgcgccccctgcccccc

SEQ ID NO: 19 - DNA, 45 bp, Homo sapiens
ctgccccccgccgccatcgagagccccgcagtggccgcgcccgcc

SEQ ID NO: 20 - DNA, 45 bp, Homo sapiens
gccatcgagagccccgcagtggccgcgcccgcctacagccgcgcg

SEQ ID NO: 21 - DNA, 45 bp, Homo sapiens
cccgcagtggccgcgcccgcctacagccgcgcgctcagccggcaa

SEQ ID NO: 22 - DNA, 45 bp, Homo sapiens
agcagcggggtctcggagatccggcacactgcggaccgctggcgc

SEQ ID NO: 23 - DNA, 45 bp, Homo sapiens
acgcagtccaacgagatcaccatcccagtcaccttcgagtcgcgg

SEQ ID NO: 24 - protein, 208 aa, Mus musculus
TERRVPFSLLRSPSWEPFRDWYPAHSRLFDQAFGVPRLPDEWSQWFSAAGWPGYVRPLPAATAEGPAAVTLAAPAFSRALNRQLSSGVSEIRQTADRWRVSLDVNHFAPEELTVKTKEGVVEITGKHEERQDEHGYISRCFTRKYTLPPGVDPTLVSSSLSPEGTLTVEAPLPKAVTQSAEITIPVTFEARAQIGGPEAGKSEQSGAK

SEQ ID NO: 25 - protein, 36 aa, Homo sapiens
MTERRVPFSLLRGPSWDPFRDWYPHSRLFDQAFGLP

SEQ ID NO: 26 - protein, 56 aa, Homo sapiens
MTERRVPFSLLRGPSWDPFRDWYPHSRLFDQAFGLPRLPEEWSQWLGGSSWPGYVR

SEQ ID NO: 27 - protein, 60 aa, Homo sapiens
MTERRVPFSLLRGPSWDPFRDWYPHSRLFDQAFGLPRLPEEWSQWLGGSSWPGYVRPLPP

SEQ ID NO: 28 - protein, 72 aa, Homo sapiens
MTERRVPFSLLRGPSWDPFRDWYPHSRLFDQAFGLPRLPEEWSQWLGGSSWPGYVRPLPPAAIESPAVAAPA

SEQ ID NO: 29 - protein, 76 aa, Homo sapiens
MTERRVPFSLLRGPSWDPFRDWYPHSRLFDQAFGLPRLPEEWSQWLGGSSWPGYVRPLPPAAIESPAVAAPAYSRA

SEQ ID NO: 30 - protein, 80 aa, Homo sapiens
MTERRVPFSLLRGPSWDPFRDWYPHSRLFDQAFGLPRLPEEWSQWLGGSSWPGYVRPLPPAAIESPAVAAPAYSRALSRQ

SEQ ID NO: 31 - protein, 96 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWR

SEQ ID NO: 32 - protein, 188 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESR

SEQ ID NO: 33 - protein, 39 aa, Homo sapiens
WSQWLGGSSWPGYVRPLPPAAIESPAVAAPAYSRALSRQ

SEQ ID NO: 34 - DNA, 108 bp, Homo sapiens
atgaccgagcgccgcgtccccttctcgctcctgcggggccccagctgggaccccttccgcgactggtacccgcatagccgcctcttcgaccaggccttcgggctgccc

SEQ ID NO: 35 - DNA, 168 bp, Homo sapiens
atgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgc

SEQ ID NO: 36 - DNA, 180 bp, Homo sapiens
atgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgcccccc

SEQ ID NO: 37 - DNA, 216 bp, Homo sapiens
atgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcc

SEQ ID NO: 38 - DNA, 270 bp, Homo sapiens
catatgcatcaccatcatcaccaccaccatgatgacgacgacaagaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcg

SEQ ID NO: 39 - DNA, 282 bp, Homo sapiens
catatgcatcaccatcatcaccaccaccatgatgacgacgacaagaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaa

SEQ ID NO: 40 - DNA, 288 bp, Homo sapiens
atgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgc

SEQ ID NO: 41 - DNA, 606 bp, Homo sapiens
catatgcatcaccatcatcaccaccaccatgatgacgacgacaagaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgg

SEQ ID NO: 42 - DNA, 114 bp, Homo sapiens
tggtcgcagtggttaggcggcagcagctggccaggctacgtgcgccccctgccccccgccgccatcgagagccccgcagtggccgcgcccgcctacagccgcgcgctcagccgg

SEQ ID NO: 43 - protein, 105 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFA

SEQ ID NO: 44 - DNA, 357 bp, Homo sapiens
catatgcatcaccatcatcaccaccaccatgatgacgacgacaagaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgcc